(19) United States
(12) Patent Application Publication
(10) Pub. No.: US 2018/0280370 A1
(43) Pub. Date: Oct. 4, 2018

(54) DOSAGE REGIMEN FOR A PHOSPHATIDYLINOSITOL 3-KINASE INHIBITOR

(71) Applicant: Novartis AG, Basel (CH)

(72) Inventors: Christian Schnell, Hegenheim (FR); Christine Fritsch, Leymen (FR); Emmanuelle di Tomaso, Lexington, MA (US); Cristian Massacesi, Neuilly Sur-Seine (FR); Lars Blumenstein, Riehen (CH)

(21) Appl. No.: 15/772,302

(22) PCT Filed: Oct. 31, 2016
(86) PCT No.: PCT/IB2016/056556

§ 371 Date: Apr. 30, 2018

Related U.S. Application Data
(60) Provisional application No. 62/249,543, filed on Nov. 2, 2015.
(60) Provisional application No. 62/393,777, filed on Sep. 13, 2016.

(51) Int. Cl. A61K 31/4439; A61K 31/5377; A61K 31/553; A61K 31/519; A61K 31/506; A61K 9/00; A61P 35/00

(52) U.S. Cl. CPC A61K 31/4439; A61K 31/5377; A61K 31/553; A61P 35/00; A61K 31/506; A61K 9/0053; A61K 31/519

(57) ABSTRACT

A method of treating or preventing a proliferative disease in a patient in need thereof by orally administering a therapeutically effective amount of a phosphatidylinositol 3-kinase inhibitor compound or a pharmaceutically acceptable salt thereof once-per-day either on a continuous daily schedule or an intermittent schedule at about zero to about three hours prior to sleep; a therapeutic regimen comprising administration of said compound or a pharmaceutically acceptable salt thereof in accordance with said dosage regimen; and related pharmaceutical compositions and packages thereof.

### FIELD OF THE DISCLOSURE

The present disclosure relates to methods of treating or preventing a proliferative disease in a patient in need thereof by orally administering a therapeutically effective amount of a phosphatidylinositol 3-kinase inhibitor compound to the patient once-per-day either on a continuous daily schedule or an intermittent schedule at about zero to about three hours prior to sleeping; the use of said phosphatidylinositol 3-kinase inhibitor for the manufacture of a medicament for treating or preventing a proliferative disease administered in accordance with said dosage regimen; a therapeutic regimen comprising administration of said phosphatidylinositol 3-kinase inhibitor in accordance with said dosage regimen; and related pharmaceutical compositions and packages thereof.

### BACKGROUND OF THE DISCLOSURE

Phosphatidylinositol 3-kinases (“PI-3 kinase” or “PI3K”) comprise a family of lipid kinases that catalyze the transfer of phosphate to the D-3′ position of inositol lipids to produce phosphoinositol-3-phosphate (“PIP”), phosphoinositol-3,4-diphosphate (“PIP2”) and phosphoinositol-3,4,5-triphosphate (“PIP3”) that, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Human cells contain three genes (PIK3CA, PIK3CB and PIK3CD) encoding the catalytic p110 subunits (α, β, δ isoforms) of class IA PI3K enzymes. These catalytic p110α, p110β, and p110δ subunits are constitutively associated with a regulatory subunit that can be p85α, p55α, p50α, p85β or p55γ, p110α, and p110β are expressed in most tissues. Class 1B PI3K has one family member, a heterodimer composed of a catalytic p110γ subunit associated with one of two regulatory subunits, either the p101 or the p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B, as well as p110δ in some circumstances, is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

PI3K inhibitors are useful therapeutic compounds for the treatment of various conditions in humans. Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers. Furthermore, mutations and translocation of p85α that serve to up-regulate the p85-p110 complex have been described in human cancers. Finally, somatic missense mutations in PIK3CA that activate downstream signaling pathways have been described at significant frequencies in a wide diversity of human cancers, including 32% of colorectal cancers, 27% of glioblastomas, 25% of gastric cancers, 36% of hepatocellular carcinomas, and 18-40% of breast cancers. (Samuels et al., Cell Cycle 3(10):1221 (2004); Hartmann et al, Acta Neuropathol., 109(6):639 (June 2005); Li et al, BMC Cancer 5:29 (March 2005); Lee et al, Oncogene, 24(8):1477 (2005); Backman et al, Cancer Biol. Ther. 3(8): 772-775 (2004); Campbell et al., Cancer Research, 64(21): 7678-7681 (2004); Levine et al., Clin. Cancer Res., 11(8): 2875-2878 (2005); and Wu et al, Breast Cancer Res., 7(5):R609-R616 (2005)). Deregulation of PI3Kis one of the most common deregulations associated with human cancers and other proliferative diseases (Parsons et al., Nature 436:792 (2005); Hennessey at el., Nature Rev. Drug Disc. 4:988-1004 (2005)).

In a Phase I clinical trial, the PI3K inhibitor compound (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) demonstrated clinical efficacy in the single-agent treatment of patients having advanced solid malignancies carrying an alteration in the PIK3CA gene. In the dose escalation phase, patients were orally administered this compound either (a) at a dosage ranging from 30 mg to 450 mg once-per 0 day (q.d.) on a continuous daily schedule for 28-days, or (b) at a dosage ranging from 120 mg to 200 mg twice per day (b.i.d.) on a continuous daily schedule for 28-days, as guided by Bayesian logistic regression model with overdose control. After determination of the maximal tolerated dose (MTD), the dose expansion phase was conducted to additionally treat patients having PIK3CA wildtype ER+/HER2-breast cancer. Clinical efficacy of this compound has been demonstrated preliminarily. As of Mar. 10, 2014, 15 of 132 evaluable patients had partial responses to treatment, and 7 were confirmed (2 at 270 mg/QD, 1 at 350 mg/QD, 2 at 400 mg/QD, and 2 at 150 mg/BID). Disease control rates (Complete response, partial response or stable disease) were 53.2% (95% CI: 40.1-66.0) and 66.7% (95% CI: 38.4-88.2) in those treated with alpelisib 400 mg/QD and 150 mg/BID, respectively. (Juric et al, “Phase I study of the PI3Kα Inhibitor BYL719, as a Single Agent in Patients with Advanced Solid Tumors (AST)”, Annals of Oncology (2014), 25 (Supp. 4): iv150.)

In a Phase I clinical trial, the PI3K inhibitor compound 4-(trifluoromethyl)-5-(2,6-dimorpholinopyrimidin-4-yl)pyridin-2-amine showed preliminary antitumor activity in patients with advanced solid tumors. Patients with advanced solid tumors (N-83) enrolled in the dose-escalation and -expansion study, and the most common cancers were colorectal (n=31) and breast cancer (n=21). One confirmed partial response (PR; triple-negative breast cancer) and three unconfirmed PRs (parotid gland carcinoma, epithelioid hemangiothelioma, ER+breast cancer) were reported. (Rodon et al., “Phase I dose-escalation and -expansion study of buparlisib (BKM120), an oral pan-Class I PI3K inhibitor, in patients with advanced solid tumors”, Invest New Drugs, 2014 August, 32(4): 670-81).

However, PI3K inhibitors may produce a negative side effect of hyperglycemia at therapeutic doses. In the Phase I clinical trials above, daily administration of (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) to human patients induced hyperglycemia in 49% of the patients. (Juric et al, Annals of Oncology (2014), 25 (Supp. 4): iv150.) In a Phase I clinical trial, daily administration of 4-(trifluoromethyl)-5-(2,6-dimorpholinopyrimidin-4-yl)pyridin-2-amine to human patients induced hyperglycemia in 31% of the patients. (Rodon et al, Invest New Drugs, 2014 August, 32(4):670-81.)

Currently, there is an unmet need for a PI3K inhibitor which can be administered to patients in a dosage or dosage regimen that is clinically effective for treatment of proliferative diseases, particularly cancer, but also that relieves, reduces, or alleviates hyperglycemia (e.g, by severity, occurrence rate, or frequency). It is believed that this has not been achieved for PI3K inhibitors prior to the present disclosure.

### SUMMARY OF THE DISCLOSURE

The present disclosure relates to a method of treating or preventing a proliferative disease in a patient in need thereof, comprising orally administering a therapeutically effective amount of a PI3K inhibitor once-per-day either on a continuous daily schedule or an intermittent schedule at about zero to about three hours prior to sleep. In a further embodiment, the phosphatidylinositol 3-kinase inhibitor is selected from the compound of formula (I)

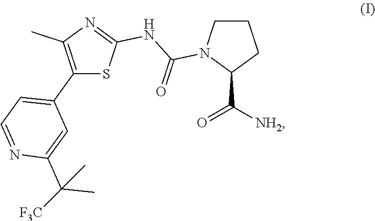 [SMILES: Cc1nc(NC(=O)N2CCC[C@H]2C(N)=O)sc1-c1ccnc(C(C)(C)C(F)(F)F)c1]

the compound of formula (II)

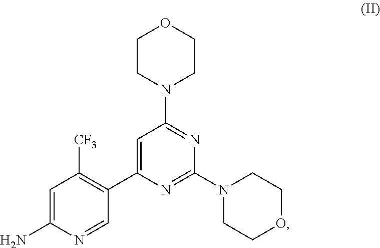 [SMILES: Nc1cc(C(F)(F)F)c(-c2cc(N3CCOCC3)nc(N3CCOCC3)n2)cn1]

pictilisib, taselisib, LY2780301, copanlisib, MLN1117, and AZD8835 ora pharmaceutically acceptable salt thereof. In one embodiment, the phosphatidylinositol 3-kinase inhibitor is the compound of formula (I)

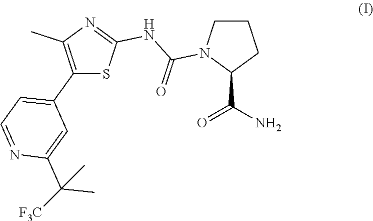 [SMILES: Cc1nc(NC(=O)N2CCC[C@H]2C(N)=O)sc1-c1ccnc(C(C)(C)C(F)(F)F)c1]

or a pharmaceutically acceptable salt thereof and administered orally in a therapeutically effective amount of about 50 mg to about 450 mg once-per-day either on a continuous daily schedule or an intermittent schedule. In another embodiment, the phosphatidylinositol 3-kinase inhibitor is the compound of formula (II)

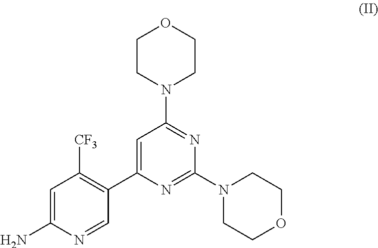 [SMILES: Nc1cc(C(F)(F)F)c(-c2cc(N3CCOCC3)nc(N3CCOCC3)n2)cn1]

or a pharmaceutically acceptable salt thereof and administered orally in a therapeutically effective amount of about 60 mg to about 120 mg once-per-day either on a continuous daily schedule or an intermittent schedule.

In a further embodiment, the phosphatidylinositol 3-kinase inhibitor is administered at about one to about two hours prior to sleep. In a still further embodiment, the phosphatidylinositol 3-kinase inhibitor is administered at night.

In another embodiment, the phosphatidylinositol 3-kinase inhibitor is administered with food at about one to three hours prior to sleep. In a further embodiment, the phosphatidylinositol 3-kinase inhibitor is administered within about zero to about one hour of ingesting food and at about one to three hours prior to sleep.

In one embodiment, the phosphatidylinositol 3-kinase inhibitor is administered on a continuous daily schedule. In another embodiment, the phosphatidylinositol 3-kinase inhibitor is administered on an intermittent schedule.

The present disclosure also relates to a method of treating or preventing a proliferative disease comprising first administering to a patient in need thereof a therapeutically effective amount of a phosphatidylinositol 3-kinase inhibitor once in each morning or twice daily; second determining said patient has a side effect of hyperglycemia after administration of said phosphatidylinositol 3-kinase inhibitor to said patient; and third shifting the administration of the phosphatidylinositol 3-kinase inhibitor to once-per-day either on a continuous daily schedule or an intermittent schedule about zero to about three hours prior to sleep.

The present disclosure also relates to the use of a phosphatidylinositol 3-kinase inhibitor, or a pharmaceutically acceptable salt thereof, for the manufacture of a medicament for treating or preventing a proliferative disease, wherein a therapeutically effective amount of said medicament is orally administered to a patient in need thereof of said phosphatidylinositol 3-kinase inhibitor at about zero to about three hours prior to sleep.

In one embodiment, the proliferative disease is a cancer. In a further embodiment, the proliferative disease is a cancer selected from a cancer of the lung (including small cell lung cancer and non-small cell lung cancer), bronchus, prostate, breast (including triple negative breast cancer, sporadic breast cancers and sufferers of Cowden disease), colon, rectum, colon carcinoma, colorectal adenoma, pancreas, gastrointestine, hepatocellular, stomach, gastric, ovary, squamous cell carcinoma, and head and neck. Preferably, the proliferative disease is breast cancer.

In one embodiment, the phosphatidylinositol 3-kinase inhibitor, or a pharmaceutically acceptable salt thereof, is administered in combination with at least one additional therapeutic agent.

The present disclosure also relates to a therapeutic regimen for the treatment or prevention of a proliferative disease comprising administering a therapeutically effective amount of a phosphatidylinositol 3-kinase inhibitor once-per-day either on a continuous daily schedule or an intermittent schedule at about zero to about three hours prior to sleep. In another embodiment, the phosphatidylinositol 3-kinase inhibitor is selected from the compound of formula (I)

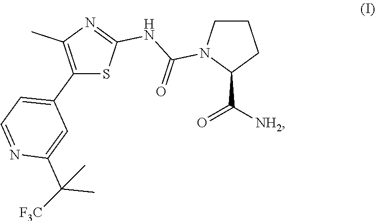 [SMILES: Cc1nc(NC(=O)N2CCC[C@H]2C(N)=O)sc1-c1ccnc(C(C)(C)C(F)(F)F)c1]

the compound of formula (II)

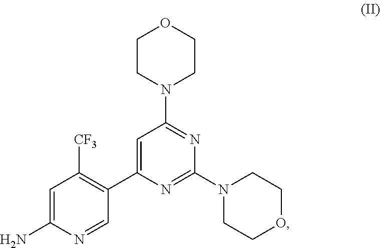 [SMILES: Nc1cc(C(F)(F)F)c(-c2cc(N3CCOCC3)nc(N3CCOCC3)n2)cn1]

pictilisib, taselisib, LY2780301, copanlisib, MLN1117, and AZD8835 ora pharmaceutically acceptable salt thereof. In one embodiment, the phosphatidylinositol 3-kinase inhibitor is the compound of formula (I)

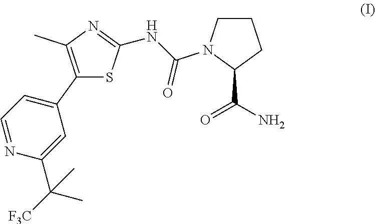 [SMILES: Cc1nc(NC(=O)N2CCC[C@H]2C(N)=O)sc1-c1ccnc(C(C)(C)C(F)(F)F)c1]

or a pharmaceutically acceptable salt thereof and administered orally in a therapeutically effective amount of about 50 mg to about 450 mg once-per-day either on a continuous daily schedule or an intermittent schedule. In another embodiment, the phosphatidylinositol 3-kinase inhibitor is the compound of formula (II)

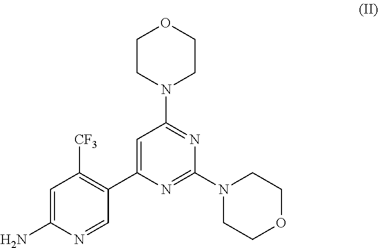 [SMILES: Nc1cc(C(F)(F)F)c(-c2cc(N3CCOCC3)nc(N3CCOCC3)n2)cn1]

or a pharmaceutically acceptable salt thereof and administered orally in a therapeutically effective amount of about 60 mg to about 120 mg once-per-day either on a continuous daily schedule or an intermittent schedule.

The present disclosure also relates to a package comprising a pharmaceutical composition comprising a phosphatidylinositol 3-kinase inhibitor together with one or more pharmaceutically acceptable excipients in combination with instructions to administer said pharmaceutical composition once-per-day either on a continuous daily schedule or an intermittent schedule at about zero to about three hours prior to sleep.

### DETAILED DESCRIPTION OF THE FIGURES

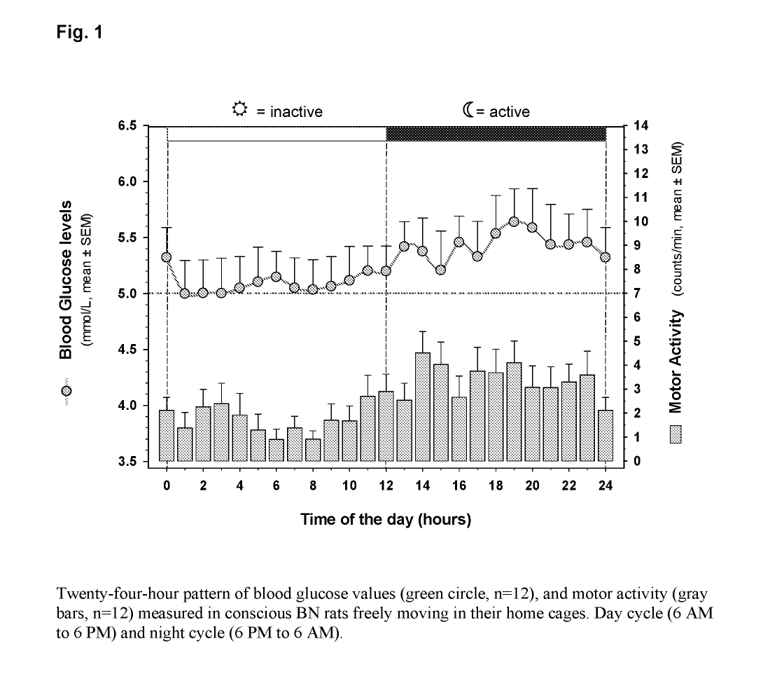

FIG. 1 shows a twenty-four-hour pattern of blood glucose values and motor activity measured in conscious Brown Norway rats freely moving in their home cages.

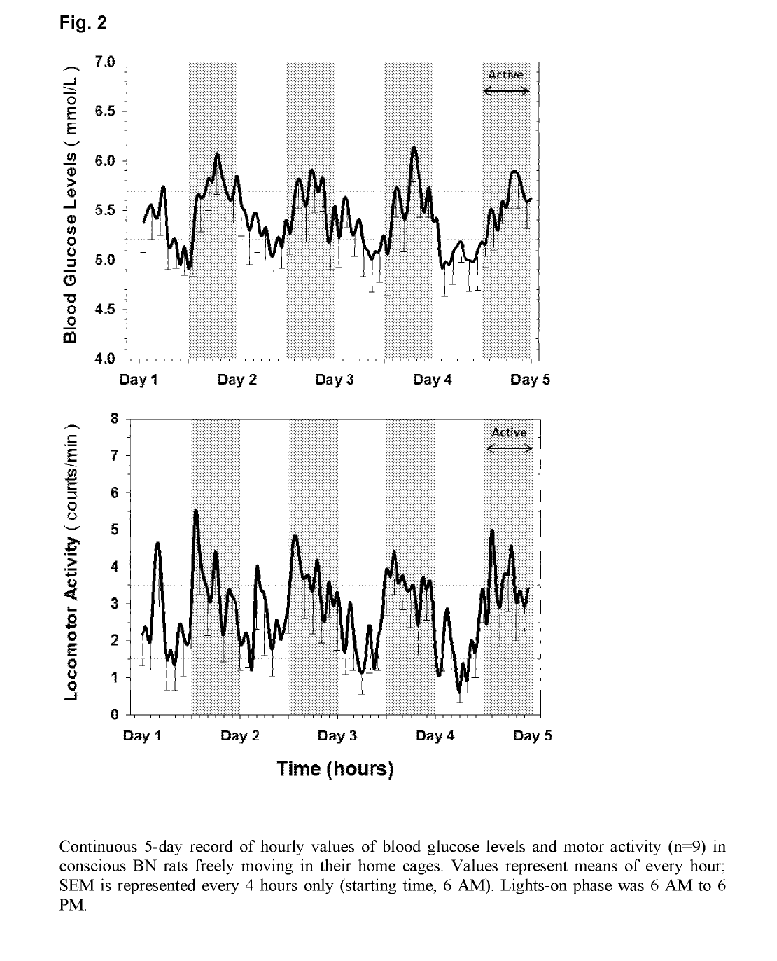

FIG. 2 shows a continuous 5-day record of hourly values of blood glucose levels and motor activity in conscious Brown Norway rats freely moving in their home cages.

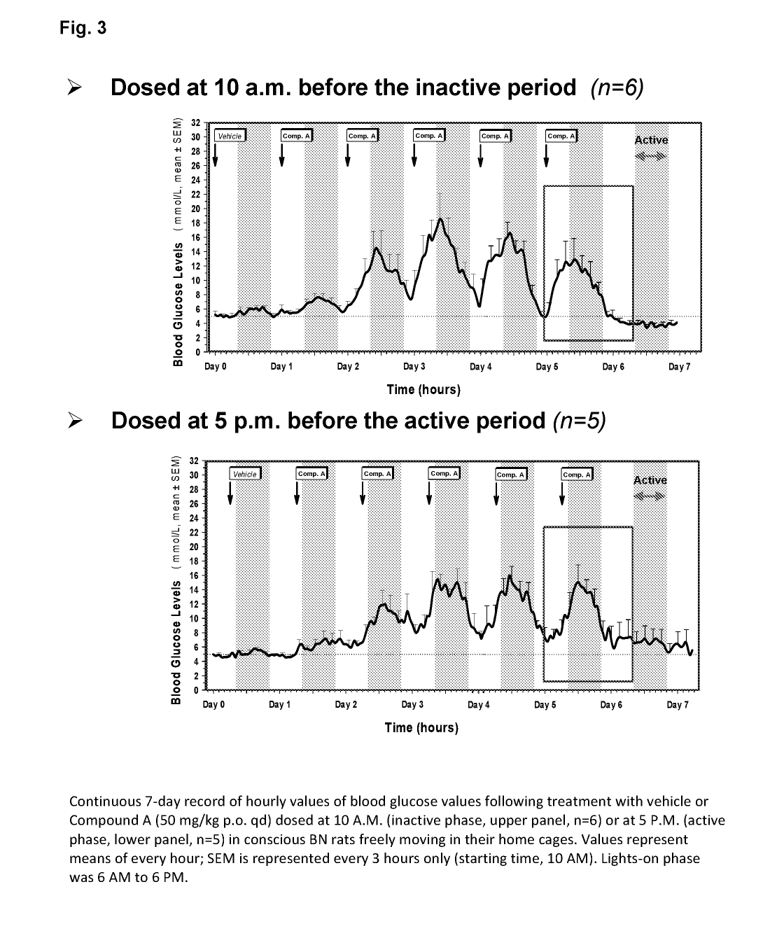

FIG. 3 shows a continuous 7-day record of hourly values of blood glucose values following treatment with vehicle or Compound A (50 mg/kg p.o. qd) dosed at 10 A.M. (inactive phase, upper panel, n=6) or at 5 P.M. (active phase, lower panel, n=5) in conscious Brown Norway rats freely moving in their home cages.

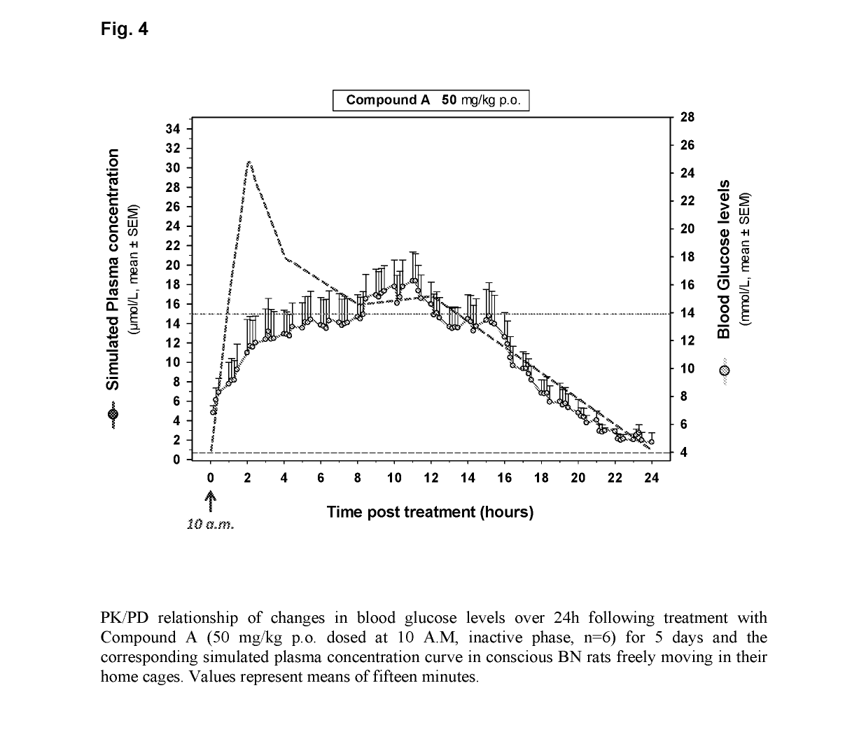

FIG. 4 shows the PK/PD relationship of changes in blood glucose levels over 24h following treatment with Compound A (50 mg/kg p.o. dosed at 10 A.M, inactive phase, n=6) for 5 days and the corresponding simulated plasma concentration curve in conscious Brown Norway rats freely moving in their home cages.

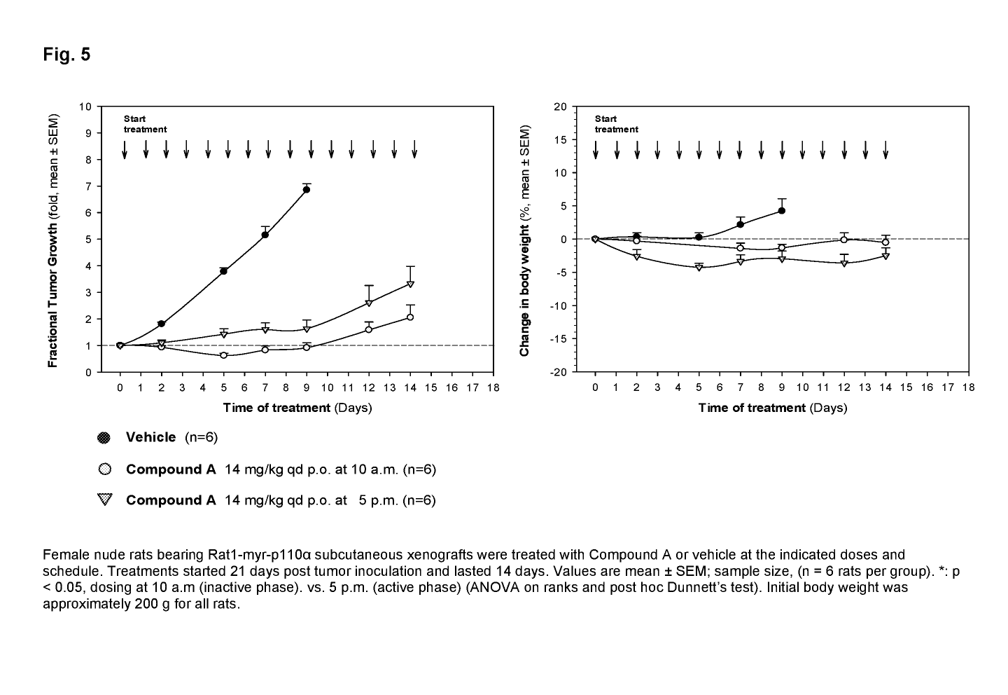

FIG. 5 shows the fractional tumor growth and change in body weight profiles for female nude rats bearing Rat1-myr-p110α subcutaneous xenografts that were treated with either Compound A (14 mg/kg) or a vehicle at the indicated doses and schedule.

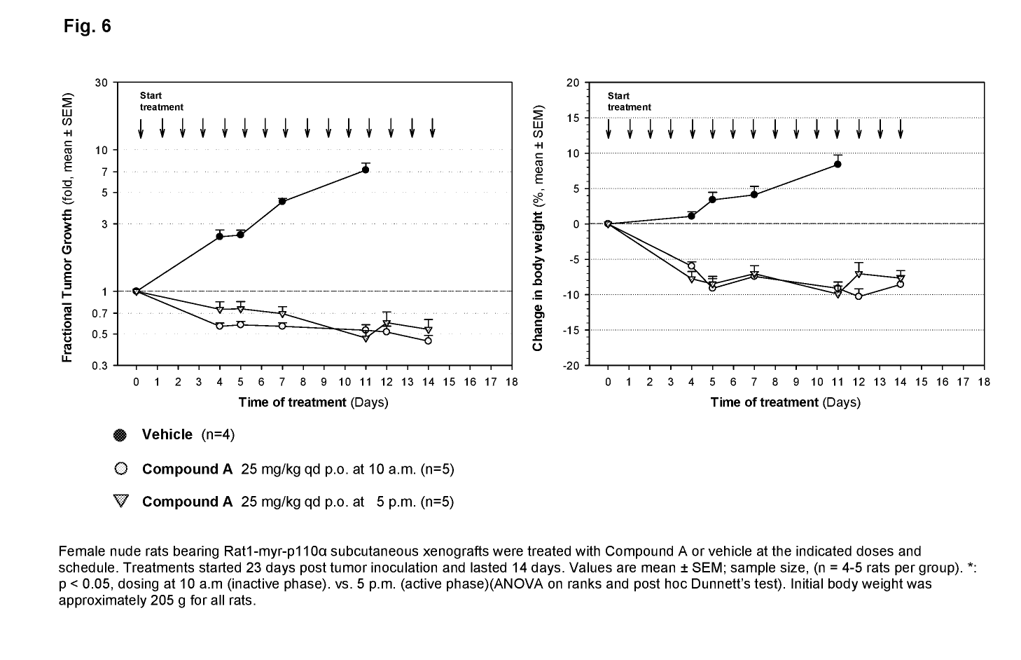

FIG. 6 shows the fractional tumor growth and change in body weight profiles for female nude rats bearing Rat1-myr-p110α subcutaneous xenografts that were treated with either Compound A (25 mg/kg) or a vehicle at the indicated doses and schedule.

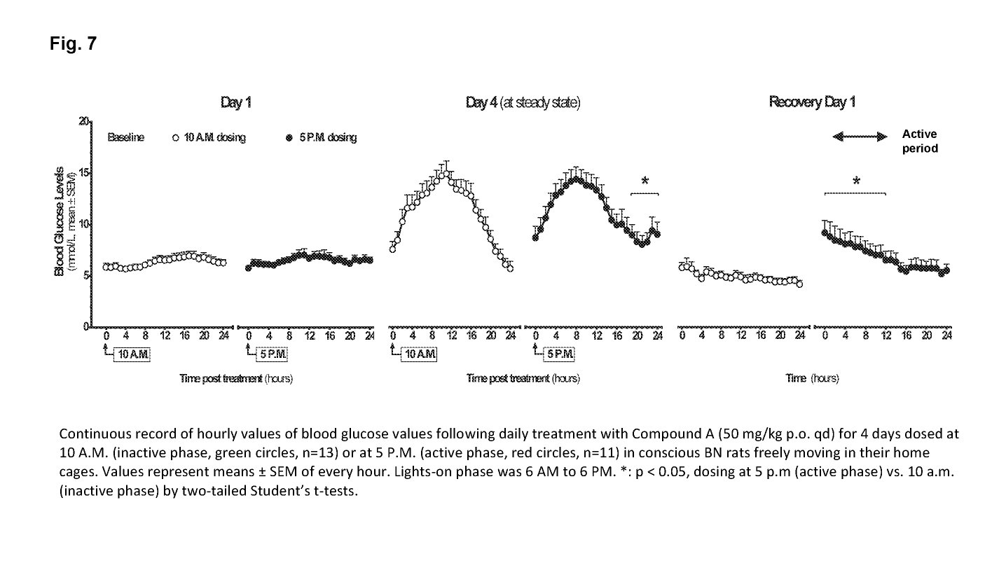

FIG. 7 shows a continuous 4-day record of hourly values of blood glucose values following daily treatment with Compound A (50 mg/kg p.o. qd) for 4 days dosed at 10 A.M. (inactive phase, white circles, n=13) or at 5 P.M. (active phase, black circles, n=11) in conscious BN rats freely moving in their home cages.

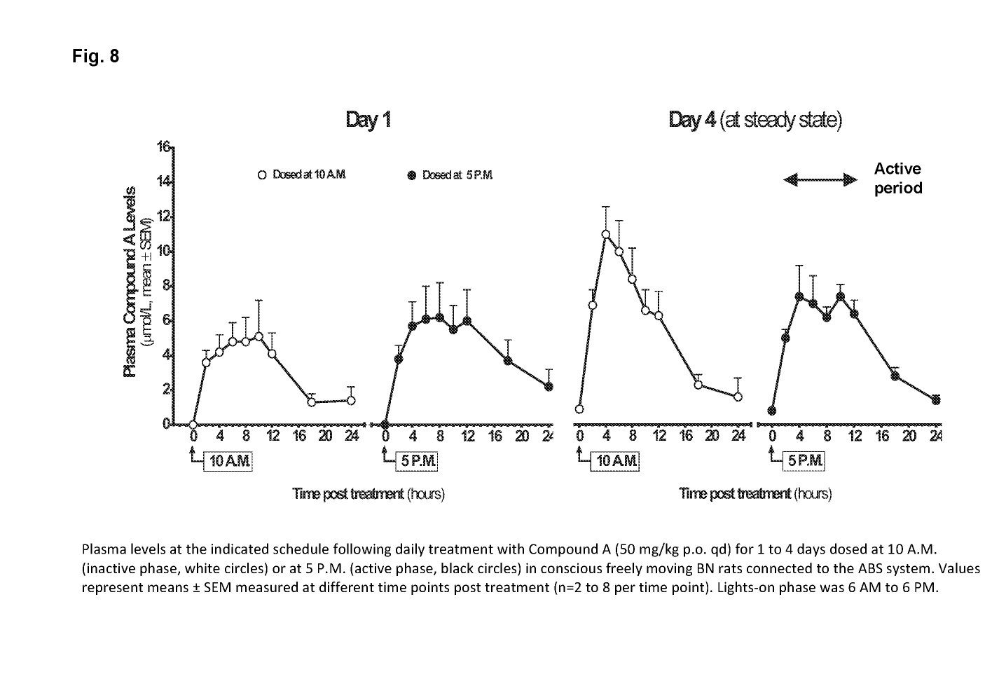

FIG. 8 shows plasma levels of Compound A at the indicated schedule following daily treatment with Compound A (50 mg/kg p.o. qd) for 1 to 4 days dosed at 10 A.M. (inactive phase, white circles) or at 5 P.M. (active phase, black circles) in conscious freely moving Brown Norway rats.

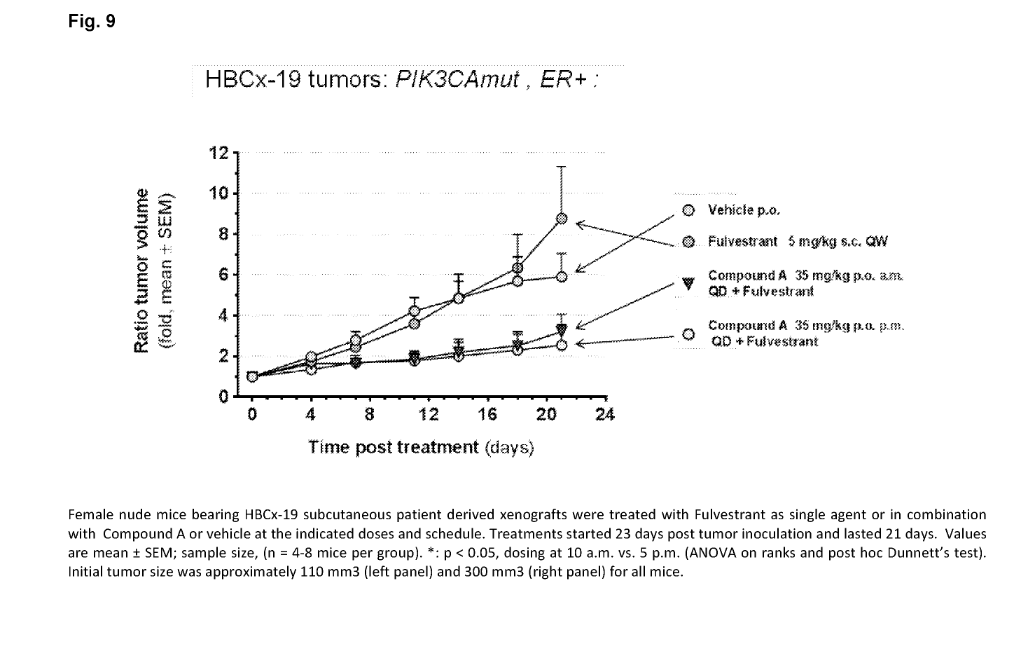

FIG. 9 shows ratio tumor volume changes for female nude mice bearing HBCx-19 subcutaneous patient derived xenografts that were treated with Fulvestrant as single agent or in combination with Compound A or vehicle at the indicated doses and schedule.

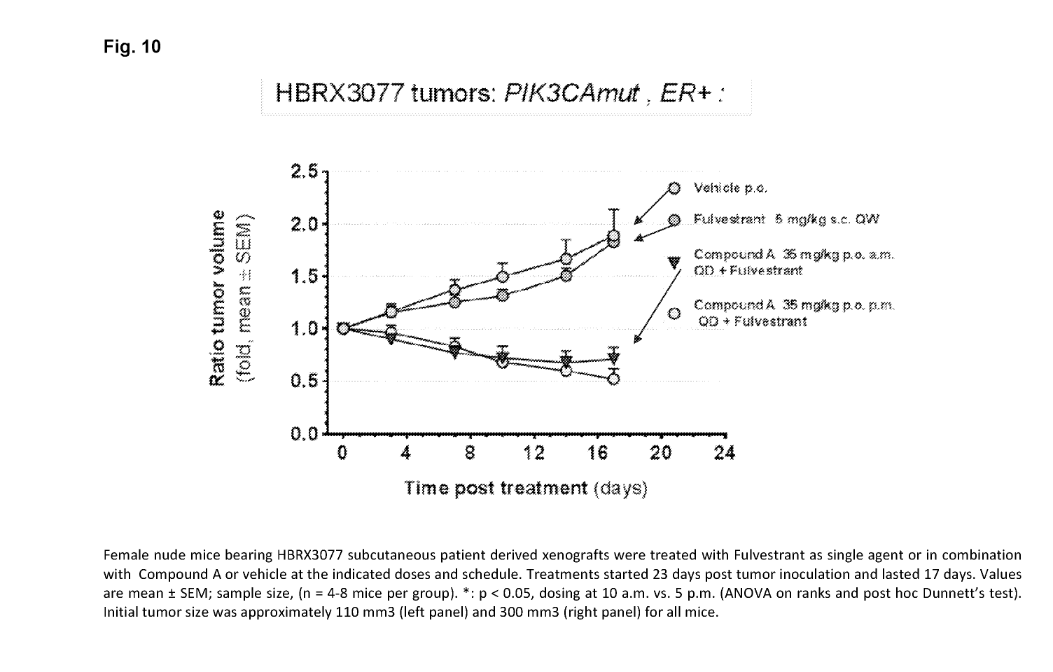

FIG. 10 shows ratio tumor volume changes for female nude mice bearing HBRX3077 subcutaneous patient derived xenografts that were treated with Fulvestrant as single agent or in combination with Compound A or vehicle at the indicated doses and schedule.

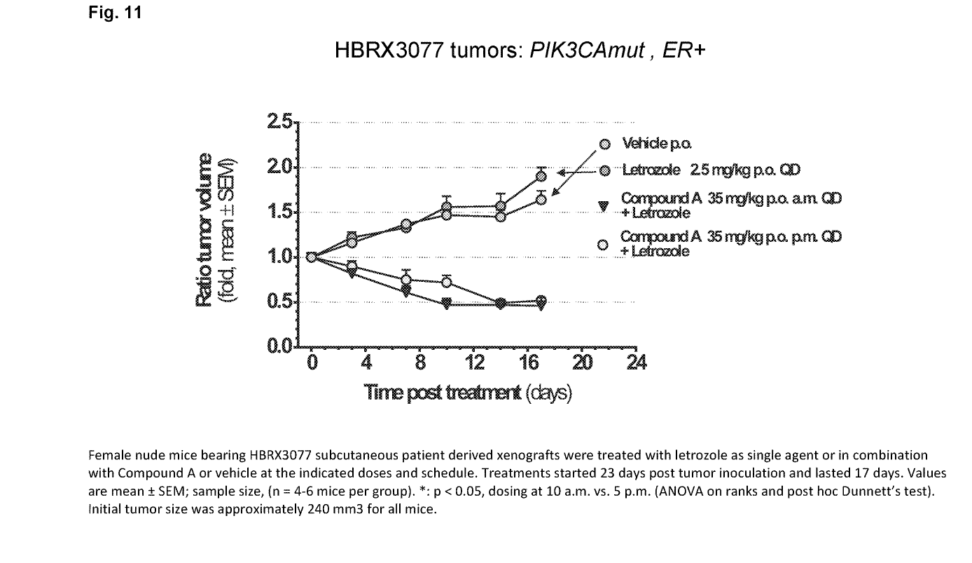

FIG. 11 shows ratio tumor volume changes for female nude mice bearing HBRX3077 subcutaneous patient derived xenografts that were treated with letrozole as single agent or in combination with Compound A or vehicle at the indicated doses and schedule.

### DETAILED DESCRIPTION OF THE DISCLOSURE

The present disclosure relates to a method of treating or preventing a proliferative disease in a patient in need thereof, comprising orally administering a therapeutically effective amount of a PI3K inhibitor once-per-day either on a continuous daily schedule or an intermittent schedule at about zero to about three hours prior to sleep. The disclosed compositions and methods provide a convenient method of administration in that a single dose can be taken typically in the evening prior to going to bed, or at whatever time of day one retires for an extended period of sleep.

Although the present compositions are described as effective as a once-a-day dosage either on a continuous daily schedule or an intermittent schedule, it is understood that additional doses can be administered as needed at the direction of a physician. The description herein is primarily directed to treatment of persons with a typical schedule of going to sleep from around 9 P.M. to about midnight, for example, and sleeping for 6-9 hours. It is understood, however, that the use and efficacy of the compositions and methods is not limited to such a schedule, but can be adopted for use with different daily schedules, such as night workers, or people with longer, shorter or more variable sleep patterns.

The general terms used herein are defined with the following meanings, unless explicitly stated otherwise:

The terms “comprising” and “including” are used herein in their open-ended and non-limiting sense unless otherwise noted.

The terms “a” and “an” and “the” and similar references in the context of describing the disclosure (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “a phosphatidylinositol 3-kinase inhibitor” or “PI3K inhibitor” is defined herein to refer to a compound which targets, decreases or inhibits activity of the phosphatidylinositol 3-kinase.

The term “pharmaceutically acceptable” is defined herein to refer to those compounds, materials, compositions and/or dosage forms, which are, within the scope of sound medical judgment, suitable for contact with the tissues a patient without excessive toxicity, irritation allergic response and other problem complications commensurate with a reasonable benefit/risk ratio.

The term “pharmaceutically acceptable salt”, as used herein, unless otherwise indicated, includes salts of acidic and basic groups which may be present in the compounds of the present invention. Such salts can be prepared in situ during the final isolation and purification of the compounds, or by separately reacting the base or acid functions with a suitable organic or inorganic acid or base, respectively. Suitable salts of the compound include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2 hydroxyethanesulfonate, lactate, maleate, methanesulfonate, nicotinate, 2 naphth-alenesulfonate, oxalate, pamoate, pectinate, persulfate, 3 phenylproionate, picrate, pivalate, propionate, succinate, sulfate, tartrate, thiocyanate, p toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others.

The term “treat”, “treating” or “treatment” as used herein comprises a treatment or therapeutic regimen relieving, reducing or alleviating at least one symptom in a patient or effecting a delay of progression of a proliferative disorder. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disorder) and/or reduce the risk of developing or worsening a disorder.

The term “prevent”, “preventing” or “prevention” as used herein comprises the prevention of at least one symptom associated with or caused by the state, disease or disorder being prevented.

The term “therapeutically effective” is an observable improvement over the baseline clinically observable signs and symptoms of the state, disease or disorder treated with the therapeutic agent.

The term “therapeutically effective amount” is an amount sufficient to provide an observable improvement over the baseline clinically observable signs and symptoms of the state, disease or disorder treated with the therapeutic agent.

The term “pharmaceutical composition” is defined herein to refer to a mixture or solution containing at least one therapeutic agent to be administered to a patient, in order to prevent or treat a particular disease or condition affecting the patient.

The phrase “continuous daily schedule” as used herein means the therapeutic agent is administered to the patient during each day for at least seven days or for an unspecified period of time or for as long as treatment is necessary. It is understood that the therapeutic agent may be administered each day in a single dosage unit or multiple dosage units.

The phrase “intermittent schedule” as used herein means the therapeutic agent is administered to the patient for a period of time and then not administered for a period of time before the same therapeutic agent is next administered to the patient. The phrase “five-consecutive day cycle” as used herein means the specified therapeutic agent is administered to the patient during each day for five-consecutive days and then not administered for a period of time before the same therapeutic agent is next administered to the patient. It is understood that the therapeutic agent may be administered each day in a single dosage unit or multiple dosage units.

The term “day” as used herein refers to either one calendar day or one 24-hour period.

The term “combination” is used herein to refer to either a fixed combination in one dosage unit form, a non-fixed combination or a kit of parts for the combined administration where the compound of formula (I) or a pharmaceutically acceptable salt thereof, and at least one additional therapeutic agent may be administered simultaneously, independently at the same time or separately within time intervals that allow that the combination partners show a cooperative, e.g., synergistic, effect. The term “fixed combination” means that the therapeutic agents, e.g. the compound of formula (I) or a pharmaceutically acceptable salt thereof and at least one additional therapeutic agent, are both administered to a patient simultaneously in the form of a single entity or dosage unit. The term “non-fixed combination” or “kit of parts” means that the therapeutic agents, e.g. the compound of formula (I) or a pharmaceutically acceptable salt thereof and at least one additional therapeutic agent, are both administered to a patient as separate entities or dosage units either simultaneously, concurrently or sequentially with no specific time limits, wherein such administration provides therapeutically effective levels of the two therapeutic agents in the body of the patient. The latter also applies to cocktail therapy, e.g. the administration of three or more therapeutic agents.

The term “combined administration” as used herein is defined to encompass the administration of the selected therapeutic agents to a single patient, and is intended to include treatment regimens in which the agents are not necessarily administered by the same route of administration or at the same time.

The terms “patient”, “subject” or “warm-blooded animal” is intended to include animals. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a brain tumor disease. Particularly preferred, the patient or warm-blooded animal is human.

The terms “about” or “approximately” usually mean within 10%, more preferably within 5%, of a given value or range.

Examples of phosphatidylinositol 3-kinanse inhibitors for use in the current invention include, but are not limited to, the compound of formula (I)

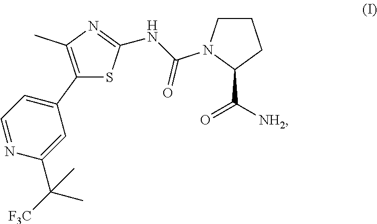 [SMILES: Cc1nc(NC(=O)N2CCC[C@H]2C(N)=O)sc1-c1ccnc(C(C)(C)C(F)(F)F)c1]

the compound of formula (II)

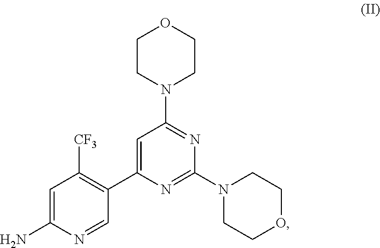 [SMILES: Nc1cc(C(F)(F)F)c(-c2cc(N3CCOCC3)nc(N3CCOCC3)n2)cn1]

pictilisib, taselisib, LY2780301, copanlisib, MLN1117, and AZD8835 ora pharmaceutically acceptable salt thereof.

WO2010/029082 describes specific 2-carboxamide cycloamino urea derivatives, which have been found to have highly selective inhibitory activity for the alpha-isoform of phosphatidylinositol 3-kinase (PI3K). A PI3K inhibitor suitable for the present invention is a compound having the following formula (I):

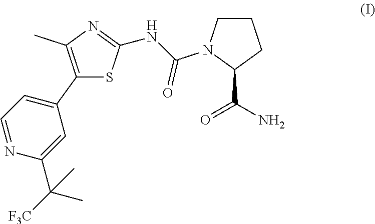 [SMILES: Cc1nc(NC(=O)N2CCC[C@H]2C(N)=O)sc1-c1ccnc(C(C)(C)C(F)(F)F)c1]

(hereinafter “compound of formula (I)” or “Compound A”) and pharmaceutically acceptable salts thereof. The compound of formula (I) is also known as the chemical compound (S)-Pyrrolidine-1, 2-dicarboxylic acid 2-amide 1-({4-methyl-5-[2-(2,2,2-trifluoro-1,1-dimethyl-ethyl)-pyridin-4-yl]-thiazol-2-yl}-amide). The compound of formula (I), its pharmaceutically acceptable salts and suitable formulations are described in PCT Application No. WO2010/029082, which is hereby incorporated by reference in its entirety, and methods of its preparation have been described, for example, in Example 15 therein. The compound of formula (I) may be present in the form of the free base or any pharmaceutically acceptable salt thereto. Preferably, compound of formula (I) is in the form of its free base.

Further, WO07/084786 describes pyrimidine derivatives, which have been found to inhibit the activity of phosphatidylinositol 3-kinase (PI3K). A PI3K inhibitor suitable for the present invention is a compound having the following formula (II)

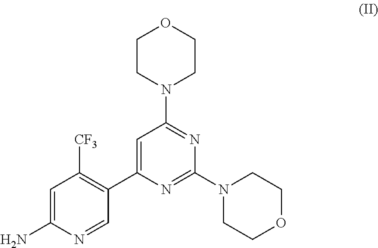 [SMILES: Nc1cc(C(F)(F)F)c(-c2cc(N3CCOCC3)nc(N3CCOCC3)n2)cn1]

(hereinafter “compound of formula (II)” or “Compound B”) and pharmaceutically acceptable salts thereof. The compound of formula (II) is also known as the chemical compound 4-(trifluoromethyl)-5-(2,6-dimorpholinopyrimidin-4-yl)pyridin-2-amine. The compound of formula (II), its pharmaceutically acceptable salts and suitable formulations are described in PCT Application No. WO07/084786, which is hereby incorporated by reference in its entirety, and methods of its preparation have been described, for example, in Example 10 therein. The compound of formula (II) may be present in the form of the free base or any pharmaceutically acceptable salt thereto. Preferably, the compound of formula (II), is in the form of its hydrochloride salt.

As used herein, the term “salts” (including “or salts thereof” or “or a salt thereof”), can be present alone or in mixture with the free base of the identified PI3K inhibitor, preferably the compound of formula (I) or the compound of formula (II) and are preferably pharmaceutically acceptable salts. For therapeutic use, only pharmaceutically acceptable salts or free compound are employed (where applicable in the form of pharmaceutical preparations), and these are therefore preferred. In view of the close relationship between the PI3K inhibitor compound in free form and those in the form of its salts, any reference to the free PI3K inhibitor herein before and hereinafter is to be understood as referring also to the corresponding salts, as appropriate and expedient.

In a preferred embodiment, the PI3K inhibitor is a compound of formula (I) or a compound of formula (II) or a pharmaceutically acceptable salt thereof.

In a preferred embodiment, the PI3K inhibitor is a compound of formula (I) or a pharmaceutically acceptable salt thereof.

The compound of formula (I) or its pharmaceutically acceptable salts may be orally administered at a therapeutically effective amount of about 50 mg to about 450 mg per day to a human patient in need thereof. In further embodiments, the compound of formula (I) may be administered to patient at a therapeutically effective amount of about 200 to about 400 mg per day, or about 240 mg to about 400 mg per day, or about 300 mg to about 400 mg per day, or about 350 mg to about 400 mg per day. In a preferred embodiment, the compound of formula (I) is administered to a human patient at a therapeutically effective amount of about 350 mg to about 400 mg per day.

The compound of formula (II) or its pharmaceutically acceptable salts may be orally administered at a therapeutically effective amount of about 60 mg to about 120 mg per day to a human patient in need thereof.

In accordance with the dosage regimen of the present disclosure, the PI3K inhibitor is orally administered to a patient in need thereof once-per-day either on a continuous daily schedule or an intermittent schedule at about zero to about three hours, e.g., about 30 minutes to about 3 hours, about 1 hour to about 3 hours, about 1 hour to about 2 hours, about 2 hours to about 3 hours, etc., prior to sleep. Preferably, the PI3K inhibitor is administered for about one to three hours prior to sleep. More preferably, the PI3K inhibitor is administered about 2 hours prior to sleep.

In one embodiment of the dosage regimen of the present disclosure, the compound of formula (I) or a pharmaceutically acceptable salt thereof is orally administered to a patient in need thereof at a therapeutically effective amount of about 100 mg to about 450 mg at about zero to about three hours prior to sleep. Preferably, the compound of formula (I) or a pharmaceutically acceptable salt thereof is administered for about one to three hours prior to sleep. More preferably, the compound of formula (I) or a pharmaceutically acceptable salt thereof is administered for about two hours prior to sleep.

In one embodiment of the dosage regimen of the present disclosure, the compound of formula (II) or a pharmaceutically acceptable salt thereof is orally administered to a patient in need thereof at a therapeutically effective amount of about 60 mg to about 120 mg at about zero to about three hours prior to sleep. Preferably, the compound of formula (II) or a pharmaceutically acceptable salt thereof is administered for about one to three hours prior to sleep. More preferably, the compound of formula (II) or a pharmaceutically acceptable salt thereof is administered for about two hours prior to sleep.

In accordance with the dosage regimen of the present disclosure, the PI3K inhibitor is orally administered to a patient in need thereof once-per-day either on a continuous daily schedule or an intermittent schedule at about zero to about three hours prior to sleep. In one embodiment, the PI3K inhibitor is orally administered to a patient in need thereof once-per-day either on a continuous daily schedule at about zero to about three hours prior to sleep. In one embodiment, the PI3K inhibitor is orally administered to a patient in need thereof once-per-day either on an intermittent schedule at about zero to about three hours prior to sleep. An example of an intermittent schedule is a five-consecutive day cycle preferably followed by a two-day period during which the therapeutic agent is not administered to the patient.

Proliferative diseases that may be treated or prevented by the administration of the compound of formula (I) or a pharmaceutically acceptable in accordance with the dosage regimen of the present disclosure. It is understood that one embodiment of the present disclosure includes the treatment of the proliferative disease and that a further embodiment of the present disclosure includes the prevention of the proliferative disease.

Examples of proliferative diseases which may be treated or prevented in accordance with the present disclosure include, cancer, myelofibrosis, haematogical disorders (e.g. haemolytic anaemia, aplastic anaemia, pure red cell anaemia and idiopathic thrombocytopenia), autoimmune inflammatory bowel disease (e.g. ulcerative colitis and Crohn's disease), Grave's disease, multiple sclerosis, uveitis (anterior and posterior), cardiovascular diseases, atherosclerosis, hypertension, deep venous thrombosis, stroke, myocardial infarction, and coronary artery disease.

Preferably, the proliferative disease is a cancer. The term “cancer” refers to tumors and/or cancerous cell growth preferably mediated by PI3K. In particular, the compounds are useful in the treatment of cancers including, for example, sarcoma, lung, bronchus, prostate, breast (including sporadic breast cancers and sufferers of Cowden disease), pancreas, gastrointestine, colon, rectum, colon carcinoma, colorectal adenoma, thyroid, liver, intrahepatic bile duct, hepatocellular, adrenal gland, stomach, gastric, glioma, glioblastoma, endometrial, melanoma, kidney, renal pelvis, urinary bladder, uterine corpus, uterine cervix, vagina, ovary, multiple myeloma, esophagus, a leukemia, acute myelogenous leukemia, chronic myelogenous leukemia, lymphocytic leukemia, myeloid leukemia, brain, oral cavity and pharynx, larynx, small intestine, non-Hodgkin lymphoma, melanoma, villous colon adenoma, a neoplasia, a neoplasia of epithelial character, lymphomas, a mammary carcinoma, basal cell carcinoma, squamous cell carcinoma, actinic keratosis, head and neck, polycythemia vera, essential thrombocythemia, myelofibrosis with myeloid metaplasia, and Waldenstroem disease.

In one embodiment, the proliferative disease is a cancer of the lung (including small cell lung cancer and non-small cell lung cancer), bronchus, prostate, breast (including triple negative breast cancer, sporadic breast cancers and sufferers of Cowden disease), colon, rectum, colon carcinoma, colorectal adenoma, pancreas, gastrointestine, hepatocellular, stomach, gastric, ovary, squamous cell carcinoma, and head and neck.

In a further embodiment, the proliferative disease is a cancer selected from a cancer of the breast, colon, rectum, colon carcinoma, colorectal adenoma, endometrial, and cervical.

In a further embodiment, the proliferative disease is a breast cancer.

In a further embodiment, the present disclosure relates to the treatment of a cancer by the administration of the compound of formula (I) or a pharmaceutically acceptable in accordance with the dosage regimen of the present disclosure.

It is believed that altering the dosing of a PI3K inhibitor compound from oral administration at (a) a daily dose prior to the patient's active phase to (b) a daily dose administered at about zero to about three hours prior to sleeping (inactive phase), is effective to treat or prevent a proliferative disease while relieving, reducing, or alleviating the severity, occurrence rate and/or frequency of any side effects. This is particularly applicable to treatment or prevention of a cancer. The term “active phase” refers to the phase in a patient's daily schedule when the patient is awake and physically active. There term “inactive phase” refers to the phase in a patient's daily schedule when the patient is sleeping for an extended period of time and not physically active.

Examples of such side effects which may be relieved, reduced, or alleviated by the dosage regimen of the present disclosure include, but are not limited to, neutropenia, elevated bilirubin, cardiac toxicity, unstable angina, myocardial infarction, persistent hypertension, peripheral sensory or motor neuropathy/pain, hepatic dysfunction (e.g., liver injury or liver disease, aspartate transaminase level elevation, alanine aminotransferase level elevation, etc.), reduced red and/or white blood cell count, hyperglycemia, nausea, decreased appetite, diarrhea, rash (e.g., maculopapular, acneiform, etc.) and hypersensitivity (e.g., increased sensitivity to bruise), photosensitivity, asthenia/fatigue, vomiting, stomatitis, oral mucositis, pancreatitis, dysgeusia, and dyspepsia. It is understood by one of ordinary skill in the art how to assess such side effects in a patient suffering from proliferative diseases using one's experience or prior knowledge and/or by referencing standard side effect grading criteria, for example, by assessing such patient using the NCI Common Terminology Criteria for Adverse Events, version 4.03 (website located at: http://evs.nci.nih.gov/ftpl/CTCAE/About.html), which is hereby incorporated by reference in its entirety.

Particularly, the side effects relieved, reduced, or alleviated by the dosage regimen of the present disclosure is hyperglycemia or rash.

It can be shown by established test models that the dosage regimen of the present disclosure results in the beneficial effects described herein before. The person skilled in the art is fully enabled to select a relevant test model to prove such beneficial effects. The pharmacological activity of the PI3K inhibitors, particularly compounds of formula (I) or (II) or their pharmaceutically acceptable salt, may, for example, be demonstrated in a clinical study, an animal study or in a test procedure as essentially described hereinafter.

Suitable clinical studies are in particular, for example, open label, dose escalation studies in patients with a proliferative disease, including for example a tumor disease, e.g., breast cancer, wherein said patients are orally administered a phosphatidylinositol 3-kinase inhibitor in accordance with the dosage regimen of the present disclosure. Preferably, patients are assigned to different groups wherein at least one group is administered the PI3K on a continuous daily schedule prior to the patients' active phase and at least one group is administered the PI3K in accordance with the dosage regimen of the present disclosure. Such studies prove in particular the efficacy of the therapeutic agent and its impact on existing or potential side effects. The beneficial effects on a proliferative disease may be determined directly through the results of these studies which are known as such to a person skilled in the art. Such studies may be, in particular, suitable to compare the effects of a continuous daily schedule using the therapeutic agents and the dosing schedule of the present disclosure. The efficacy of the treatment may be determined in such studies, e.g., after 12, 18 or 24 weeks by evaluation of glucose levels, symptom scores and/or tumor size measurements every 6 weeks.

In accordance with the present disclosure, the PI3K is preferably used or administered in the form of pharmaceutically compositions that contain a therapeutically effective amount of the PI3K together with one or more pharmaceutically acceptable excipients suitable for oral administration.

In one embodiment, the compound of formula (I) or a pharmaceutically acceptable salt thereof is preferably used or administered in the form of pharmaceutically compositions that contain a therapeutically effective amount of the compound of formula (I) or pharmaceutically acceptable salt thereof together with one or more pharmaceutically acceptable excipients suitable for oral administration. The pharmaceutical composition may comprise an amount of about 100 mg to about 450 mg of a compound of formula (I) or pharmaceutically acceptable salt thereof to be administered in a single dosage unit. Alternatively, the pharmaceutical composition may comprise an amount of the compound of formula (I) or pharmaceutically acceptable salt thereof which is subdivided into multiple dosage units and administered for a therapeutically effective amount of about 50 mg to about 450 mg of the compound of formula (I) or pharmaceutically acceptable salt thereof.

In another embodiment, the compound of formula (II) or a pharmaceutically acceptable salt thereof is preferably used or administered in the form of pharmaceutically compositions that contain a therapeutically effective amount of the compound of formula (II) or pharmaceutically acceptable salt thereof together with one or more pharmaceutically acceptable excipients suitable for oral administration. The pharmaceutical composition may comprise an amount of about 60 mg to about 120 mg of a compound of formula (II) or pharmaceutically acceptable salt thereof to be administered in a single dosage unit. Alternatively, the pharmaceutical composition may comprise an amount of the compound of formula (II) or pharmaceutically acceptable salt thereof which is subdivided into multiple dosage units and administered for a therapeutically effective amount of about 60 mg to about 120 mg of the compound of formula (II) or pharmaceutically acceptable salt thereof.

The pharmaceutical compositions used according to the present disclosure can be prepared in a manner known per se to be suitable for oral administration to mammals (warm-blooded animals), including humans. Pharmaceutical compositions for oral administration may include, for example, those in dosage unit forms, such as sugar-coated tablets, tablets, capsules, sachets and furthermore ampoules. If not indicated otherwise, these are prepared in a manner known per se, for example by means of conventional mixing, granulating, sugar-coating, dissolving or lyophilizing processes. It will be appreciated that the amount of the active ingredient contained in an individual dose or dosage unit need not in itself constitute a therapeutically effective amount since the necessary effective amount can be reached by administration of a plurality of dosage units.

The novel pharmaceutical composition may contain, for example, from about 10% to about 100%, preferably from about 20% to about 60%, of the active ingredient.

In preparing the compositions for oral dosage unit form, any of the usual pharmaceutically acceptable excipients may be employed, such as, for example, water, glycols, oils, alcohols, flavoring agents, preservatives, coloring agents; or excipients such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

One of ordinary skill in the art may select one or more of the aforementioned excipients with respect to the particular desired properties of the dosage unit form by routine experimentation and without any undue burden. The amount of each excipient used may vary within ranges conventional in the art. The following references which are all hereby incorporated by reference disclose techniques and excipients used to formulate oral dosage forms. (See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003).)

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

A dosage unit form containing the compound of formula (I) or a pharmaceutically acceptable salt thereof may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose, Sodium stearyl fumarate and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

In a further embodiment, the present disclosure relates to a method of reducing at least one side effect selected from neutropenia, elevated bilirubin, cardiac toxicity, unstable angina, myocardial infarction, persistent hypertension, peripheral sensory or motor neuropathy/pain, hepatic dysfunction (e.g., liver injury or liver disease, aspartate transaminase level elevation, alanine aminotransferase level elevation, etc.), reduced red and/or white blood cell count, hyperglycemia, nausea, decreased appetite, diarrhea, rash (e.g., maculopapular, acneiform, etc.) and hypersensitivity (e.g., increased sensitivity to bruise), photosensitivity, asthenia/fatigue, vomiting, stomatitis, oral mucositis, pancreatitis, dysgeusia, and dyspepsia from prior treatment with a phosphatidylinositol 3-kinase inhibitor comprising orally administering a therapeutically effective amount of the a phosphatidylinositol 3-kinase inhibitor to the patient in a therapeutically effective amount of about 100 mg to about 450 mg, preferably about 200 mg to about 400 mg or more preferably about 350 mg to about 400 mg, once-per-day either on a continuous daily schedule or an intermittent schedule at about zero to about three hours prior to sleep. Preferably, the side effect is hyperglycemia. In another embodiment, the side effect is rash.

Further, the present disclosure includes a method of treating or preventing a proliferative disorder in accordance with any other embodiment disclosed above for the present disclosure.

In one embodiment, the present disclosure relates to the use of a phosphatidylinositol 3-kinase inhibitor for the manufacture of a medicament for treating or preventing a proliferative disease, wherein a therapeutically effective amount of said medicament is orally administered to a patient in need thereof of said phosphatidylinositol 3-kinase inhibitor once-per-day either on a continuous daily schedule or an intermittent schedule at about zero to about three hours prior to sleep.

Further, the present disclosure includes any use of the compound of formula (I) or a pharmaceutically acceptable salt thereof in accordance with the methods of treatment, uses for the manufacture of a medicament, or any embodiment disclosed above for the present disclosure.

Still further, the present disclosure includes any use of the compound of formula (II), or a pharmaceutically acceptable salt thereof in accordance with the methods of treatment, uses for the manufacture of a medicament, or any embodiment disclosed above for the present disclosure.

The present disclosure further relates to a therapeutic regimen comprising orally administering a therapeutically effective amount of a phosphatidylinositol 3-kinase inhibitor to a patient in need thereof once-per-day either on a continuous daily schedule or an intermittent schedule at about zero to about three hours prior to sleep. In one embodiment, the phosphatidylinositol 3-kinase inhibitor is the compound of formula (I), or a pharmaceutically acceptable salt thereof is administered to a patient in need thereof in a therapeutically effective amount of about 50 mg to about 450 mg. In one embodiment, the phosphatidylinositol 3-kinase inhibitor is the compound of formula (II), or a pharmaceutically acceptable salt thereof is administered to a patient in need thereof in a therapeutically effective amount of about 60 mg to about 120 mg.

The present disclosure further relates to the phosphatidylinositol 3-kinase inhibitor administered in combination with at least one additional therapeutic agent for the treatment or prevention of a proliferative disease, wherein the phosphatidylinositol 3-kinase inhibitor is administered once-per-day either on a continuous daily schedule or an intermittent schedule at about zero to about three hours prior to sleep. In one embodiment, the compound of formula (I) or a pharmaceutically acceptable salt thereof is administered in combination with at least one additional therapeutic agent for the treatment or prevention of a proliferative disease, wherein the compound of formula (I) or a pharmaceutically acceptable salt thereof is administered in a therapeutically effective amount of about 50 mg to about 450 mg once a day either on a continuous daily schedule or an intermittent schedule at about zero to about three hours prior to sleep. In another embodiment, the compound of formula (II) or a pharmaceutically acceptable salt thereof is administered in combination with at least one additional therapeutic agent for the treatment or prevention of a proliferative disease, wherein the compound of formula (II) or a pharmaceutically acceptable salt thereof is administered in a therapeutically effective amount of about 60 mg to about 120 mg once-per-day either on a continuous daily schedule or an intermittent schedule at about zero to about three hours prior to sleep.

Suitable therapeutic agents for use in accordance with the present disclosure include, but are not limited to, kinase inhibitors, anti-estrogens, anti androgens, other inhibitors, cancer chemotherapeutic drugs, alkylating agents, chelating agents, biological response modifiers, cancer vaccines, agents for antisense therapy. Examples are set forth below:

A. Kinase Inhibitors including inhibitors of Epidermal Growth Factor Receptor (EGFR) kinases such as small molecule quinazolines, for example gefitinib (U.S. Pat. No. 5,457,105, U.S. Pat. No. 5,616,582, and U.S. Pat. No. 5,770,599), ZD-6474 (WO 01/32651), erlotinib (Tarceva®, U.S. Pat. No. 5,747,498 and WO 96/30347), and lapatinib (U.S. Pat. No. 6,727,256 and WO 02/02552), and cetuximab; Vascular Endothelial Growth Factor Receptor (VEGFR) kinase inhibitors, including SU-11248 (WO 01/60814), SU 5416 (U.S. Pat. No. 5,883,113 and WO 99/61422), SU 6668 (U.S. Pat. No. 5,883,113 and WO 99/61422), CHIR-258 (U.S. Pat. No. 6,605,617 and U.S. Pat. No. 6,774,237), vatalanib or PTK-787 (U.S. Pat. No. 6,258,812), VEGF-Trap (WO 02/57423), B43-Genistein (WO-09606116), fenretinide (retinoic acid p-hydroxyphenylamine) (U.S. Pat. No. 4,323,581), IM-862 (WO 02/62826), bevacizumab or Avastin® (WO 94/10202), KRN-951, 3-[5-(methylsulfonylpiperadine methyl)-indolyl]-quinolone, AG-13736 and AG-13925, pyrrolo[2,1-f][1,2,4]triazines, ZK-304709, Veglin®, VMDA-3601, EG-004, CEP-701 (U.S. Pat. No. 5,621,100), Candy (WO 04/09769); Erb2 tyrosine kinase inhibitors such as pertuzumab (WO 01/00245), trastuzumab, and rituximab; Akt protein kinase inhibitors, such as RX-0201; Protein Kinase C (PKC) inhibitors, such as LY-317615 (WO 95/17182), and perifosine (US 2003171303); Raf/Map/MEK/Ras kinase inhibitors including sorafenib (BAY 43-9006), ARQ-350RP, LErafAON, BMS-354825 AMG-548, MEK162, and others disclosed in WO 03/82272; Fibroblast Growth Factor Receptor (FGFR) kinase inhibitors; Cell Dependent Kinase (CDK) inhibitors, including CYC-202, roscovitine (WO 97/20842 and WO 99/02162), or 7-Cyclopentyl-2-(5-piperazin-1-yl-pyridin-2-ylamino)-7H-pyrrolo[2,3-d]pyrimidine-6-carboxylic acid dimethylamide (also known as “LEE011” or “ribociclib”)(WO2010/020675 in example 74); Platelet-Derived Growth Factor Receptor (PDGFR) kinase inhibitors such as CHIR-258, 3G3 mAb, AG-13736, SU-11248 and SU6668; and Bcr-Abl kinase inhibitors and fusion proteins such as STI-571 or Gleevec® (imatinib).

B. Anti-Estrogens: Estrogen-targeting agents include Selective Estrogen Receptor Modulators (SERMs) including tamoxifen, toremifene, raloxifene; aromatase inhibitors including Arimidex® or anastrozole; Estrogen Receptor Downregulators (ERDs) including Faslodex® or fulvestrant.

C. Anti-Androgens: Androgen-targeting agents including flutamide, bicalutamide, finasteride, aminoglutethamide, ketoconazole, and corticosteroids.

D. Other Inhibitors including Protein farnesyl transferase inhibitors including tipifarnib or R-115777 (US 2003134846 and WO 97/21701), BMS-214662, AZD-3409, and FTI-277; topoisomerase inhibitors including merbarone and diflomotecan (BN-80915); mitotic kinesin spindle protein (KSP) inhibitors including SB-743921 and MKI-833; proteasome modulators such as bortezomib or Velcade® (U.S. Pat. No. 5,780,454), XL-784; cyclooxygenase 2 (COX-2) inhibitors including non-steroidal antiinflammatory drugs I (NSAIDs); letrozole; exemestane; and eribulin.

E. Cancer Chemotherapeutic Drugs including anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

F. Alkylating Agents including VNP-40101 M or cloretizine, oxaliplatin (U.S. Pat. No. 4,169,846, WO 03/24978 and WO 03/04505), glufosfamide, mafosfamide, etopophos (U.S. Pat. No. 5,041,424), prednimustine; treosulfan; busulfan; irofluven (acylfulvene); penclomedine; pyrazoloacridine (PD-115934); 06-benzylguanine; decitabine (5-aza-2-deoxycytidine); brostallicin; mitomycin C (MitoExtra); TLK-286 (Telcyta®); temozolomide; trabectedin (U.S. Pat. No. 5,478,932); AP-5280 (Platinate formulation of Cisplatin); porfiromycin; and clearazide (meclorethamine).

G. Chelating Agents including tetrathiomolybdate (WO 01/60814); RP-697; Chimeric T84.66 (cT84.66); gadofosveset (Vasovist®); deferoxamine; and bleomycin optionally in combination with electorporation (EPT).

H. Biological Response Modifiers, such as immune modulators, including staurosprine and macrocyclic analogs thereof, including UCN-01, CEP-701 and midostaurin (see WO 02/30941, WO 97/07081, WO 89/07105, U.S. Pat. No. 5,621,100, WO 93/07153, WO 01/04125, WO 02/30941, WO 93/08809, WO 94/06799, WO 00/27422, WO 96/13506 and WO 88/07045); squalamine (WO 01/79255); DA-9601 (WO 98/04541 and U.S. Pat. No. 6,025,387); alemtuzumab; interferons (e.g. IFN-a, IFN-b etc.); interleukins, specifically IL-2 or aldesleukin as well as IL-1, IL-3, IL-4, IL-5, IL-6, IL-7, IL-8, IL-9, IL-10, IL-11, IL-12, and active biological variants thereof having amino acid sequences greater than 70% of the native human sequence; altretamine (Hexalen®); SU 101 or leflunomide (WO 04/06834 and U.S. Pat. No. 6,331,555); imidazoquinolines such as resiquimod and imiquimod (U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612); and SMIPs, including benzazoles, anthraquinones, thiosemicarbazones, and tryptanthrins (WO 04/87153, WO 04/64759, and WO 04/60308).

I. Cancer Vaccines: Anticancer vaccines including Avicine® (Tetrahedron Lett. 26:2269-70 (1974)); oregovomab (OvaRex®); Theratope® (STn-KLH); Melanoma Vaccines; GI-4000 series (GI-4014, GI-4015, and GI-4016), which are directed to five mutations in the Ras protein; GlioVax-1; MelaVax; Advexin® or INGN-201 (WO 95/12660); Sig/E7/LAMP-1, encoding HPV-16 E7; MAGE-3 Vaccine or M3TK (WO 94/05304); HER-2VAX; ACTIVE, which stimulates T-cells specific for tumors; GM-CSF cancer vaccine; and Listeria monocytogenes-based vaccines.

J. Antisense Therapy: Anticancer agents including antisense compositions, such as AEG-35156 (GEM-640); AP-12009 and AP-11014 (TGF-beta2-specific antisense oligonucleotides); AVI-4126; AVI-4557; AVI-4472; oblimersen (Genasense®); JFS2; aprinocarsen (WO 97/29780); GTI-2040 (R2 ribonucleotide reductase mRNA antisense oligo) (WO 98/05769); GTI-2501 (WO 98/05769); liposome-encapsulated c-Raf antisense oligodeoxynucleotides (LErafAON) (WO 98/43095); and Sirna-027 (RNAi-based therapeutic targeting VEGFR-1 mRNA).

In one embodiment, the additional therapeutic agent is selected from gefinitib, erlotinib, bevacizumab or Avastin®, pertuzumab, trastuzumab, MEK162, tamoxifen, fulvestrant, capecitabine, cisplatin, carboplatin, cetuximab, paclitaxel, temozolamide, letrozole, everolimus or Affinitor®, 7-Cyclopentyl-2-(5-piperazin-1-yl-pyridin-2-ylamino)-7H-pyrrolo[2,3-d]pyrimidine-6-carboxylic acid dimethylamide, or exemestane.

In a further embodiment, Compound A is administered in combination with 7-Cyclopentyl-2-(5-piperazin-1-yl-pyridin-2-ylamino)-7H-pyrrolo[2,3-d]pyrimidine-6-carboxylic acid dimethylamide. In another embodiment, Compound A is administered in combination with paclitaxel. In another embodiment, Compound A is administered in combination with letrozole. In another embodiment, Compound A is administered in combination with fulvestrant. In another embodiment, Compound A is administered in combination with everolimus.

In a further embodiment, Compound B is administered in combination with 7-Cyclopentyl-2-(5-piperazin-1-yl-pyridin-2-ylamino)-7H-pyrrolo[2,3-d]pyrimidine-6-carboxylic acid dimethylamide. In still another embodiment, Compound B is administered in combination with paclitaxel. In another embodiment, Compound B is administered in combination with letrozole. In another embodiment, Compound B is administered in combination with fulvestrant. In another embodiment, Compound B is administered in combination with everolimus.

The structure of the drug substances identified by code numbers, generic or trade names may be taken from the Internet, actual edition of the standard compendium “The Merck Index” or from databases, e.g., Patents International, e.g., IMS World Publications, or the publications mentioned above and below. The corresponding content thereof is hereby incorporated by reference.

The phosphatidylinositol 3-kinase inhibitor and the additional therapeutic agent may be administered together in a single pharmaceutical composition, separately in two or more separate unit dosage forms, or sequentially. The pharmaceutical composition or dosage unit form comprising the additional therapeutic agent may be prepared in a manner known per se and are those suitable for enteral, such as oral or rectal, topical, and parenteral administration to subjects, including mammals (warm-blooded animals) such as humans.

In particular, a therapeutically effective amount of each of the therapeutic agents may be administered simultaneously or sequentially and in any order, and the components may be administered separately or as a fixed combination. For example, the combination of the present disclosure may comprise: (i) administration of the first therapeutic agent (a) in free or pharmaceutically acceptable salt form; and (ii) administration of an therapeutic agent (b) in free or pharmaceutically acceptable salt form, simultaneously or sequentially in any order, in jointly therapeutically effective amounts, preferably in synergistically effective amounts, e.g., in daily or intermittent dosages corresponding to the amounts described herein. The individual therapeutic agents of the combination may be administered separately at different times during the course of therapy or concurrently in divided or single combination forms.

“Synergy” or “synergistic” refers to the action of two therapeutic agents such as, for example, (a) a compound of formula (I) or a pharmaceutically acceptable salt thereof and (b) an aromatase inhibitor, producing an effect, for example, slowing the symptomatic progression of a cancer disease or disorder, particularly cancer, or symptoms thereof, which is greater than the simple addition of the effects of each therapeutic agent administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talelay, P., Adv. Enzyme Regul. 22: 27-55 (1984)). Each equation referred to above can be applied to experimental data to generate a corresponding graph to aid in assessing the effects of the therapeutic agent combination. The corresponding graphs associated with the equations referred to above are the concentration-effect curve, isobologram curve and combination index curve, respectively. Synergy may be further shown by calculating the synergy score of the combination according to methods known by one of ordinary skill.

The effective dosage of each of therapeutic agent (a) or therapeutic agent (b) employed in the combination may vary depending on the particular compound or pharmaceutical composition employed, the mode of administration, the condition being treated, and the severity of the condition being treated. Thus, the dosage regimen of the combination is selected in accordance with a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the therapeutic agent required to prevent, counter or arrest the progress of the condition. Optimal precision in achieving concentration of therapeutic agent within the range that yields efficacy requires a regimen based on the kinetics of the therapeutic agent's availability to target sites. This involves a consideration of the distribution, equilibrium, and elimination of a therapeutic agent.

Examples of proliferative diseases that may be treated with a combination of a compound of formula (I) or a pharmaceutically acceptable salt thereof and at least one additional therapeutic agent include, but not limited to, those set forth above.

It can be shown by established test models that the combination of the present disclosure results in the beneficial effects described herein before. The person skilled in the art is fully enabled to select a relevant test model to prove such beneficial effects. The pharmacological activity of a combination of the present disclosure may, for example, be demonstrated in a clinical study or in a test procedure as essentially described hereinafter.

Suitable clinical studies are in particular, for example, open label, dose escalation studies in patients with a proliferative disease, including for example a tumor disease, e.g., breast cancer. Such studies prove in particular the synergism of the therapeutic agents of the combination of the present disclosure. The beneficial effects on a proliferative disease may be determined directly through the results of these studies which are known as such to a person skilled in the art. Such studies may be, in particular, suitable to compare the effects of a monotherapy using the therapeutic agents and a combination of the present disclosure. In one embodiment, the dose of the PI3K inhibitor compound of formula (I) or its pharmaceutically acceptable salt is escalated until the Maximum Tolerated Dosage is reached, and the combination partner is administered with a fixed dose. Alternatively, the compound of formula (I) or its pharmaceutically acceptable salt may be administered in a fixed dose and the dose of the combination partner may be escalated. Each patient may receive doses of the compound of formula (I) or its pharmaceutically acceptable salt either once-per-day either on a continuous daily schedule or an intermittent schedule or more than once (e.g., twice) per day. The efficacy of the treatment may be determined in such studies, e.g., after 12, 18 or 24 weeks by evaluation of symptom scores every 6 weeks.

In one embodiment, the present disclosure relates to a method of treating or preventing a proliferative disease by administration in accordance with the dosage regimen of the present disclosure, wherein said phosphatidylinositol 3-kinase inhibitor is administered in combination with at least one additional therapeutic agent.

In a further embodiment, the present disclosure relates to the use of the compound of formula (I) or a pharmaceutically acceptable salt thereof for the manufacture of a medicament for treating or preventing a proliferative disease in accordance with the dosage regimen of the present disclosure, wherein said phosphatidylinositol 3-kinase inhibitor is administered in combination with at least one additional therapeutic agent.

In a further embodiment, the present disclosure relates to the use of the compound of formula (I) or a pharmaceutically acceptable salt thereof for treating or preventing a proliferative disease in accordance with the dosage regimen of the present disclosure, wherein said phosphatidylinositol 3-kinase inhibitor is administered in combination with at least one additional therapeutic agent.

The present disclosure further relates to a package comprising a pharmaceutical composition comprising a phosphatidylinositol 3-kinase inhibitor with one or more pharmaceutically acceptable excipients in combination with instructions to orally administer said pharmaceutical composition once-per-day either on a continuous daily schedule or an intermittent schedule at about zero to about three hours prior to sleep. In one embodiment, the phosphatidylinositol 3-kinase inhibitor is the compound of formula (I) or a pharmaceutically acceptable salt thereof in a dose of about 50 mg to about 450 mg. In another embodiment, the phosphatidylinositol 3-kinase inhibitor is the compound of formula (II) or a pharmaceutically acceptable salt thereof in a dose of about 60 mg to about 120 mg.

Utility of the dosage regimen of the compounds of formula (I) of the present disclosure may be demonstrated in animal test methods as well as in clinic studies. For example in the utility of the compounds of formula (I) in accordance with the present disclosure may be demonstrated in accordance with the methods hereinafter described:

### Example 1

### Materials and Methods

Animals and maintenance conditions: Experiments were performed in female nude Rowett rats Hsd: RH-Fox1rnu or female Brown-Norway (BN) rats (Harlan (The Netherlands). Animals were 6-9 weeks of age at time of application of the compound. Animals were housed under Optimized Hygienic Conditions in Makrolon type III cages (max. 2 animals per cage) with free access to food and water. They were allowed to adapt for at least 6 days before the experiment was started.

Cell line and cell culture: Rat1-Myr-p110α cells were grown in Dulbecco's Modified Eagle Medium (DMEM) culture medium containing 4.5 g/l glucose supplemented with 10% heat-inactivated fetal calf serum (FCS), 2 mM L-glutamine, 1 mM sodium pyruvate and incubated at 37° C. in a 5% CO₂ humidified atmosphere. Cells were harvested with trypsin-EDTA, re-suspended in culture medium (with additives) and counted with a Casy® system. Finally, cells were centrifuged, suspended in ice-cold Hanks' balanced salt solution (HBSS) at a concentration of 3×10⁷ cells/ml. Cell culture reagents were purchased from BioConcept (Allschwil, Switzerland).

Rat1-myr-p110α cells were generated by the method described in Maira et al., Molecular Cancer Therapeutics, 11:317-328 (2012), which is incorporated herein by reference in its entirety. Briefly, Rat1 cells were transfected to stably express the constitutively active form of the catalytic PI3K class I p110 isoforms a by addition of a myristylation signal to the N-terminus.

Establishment of Tumor Xenografts In Vivo:

Rat1-Myr-p110α tumors were established by subcutaneous injection of 5×10⁶ cells in 100 μL HBSS (Sigma #H8264) into the right flank of nude rats. For the efficacy experiments, treatments were initiated when the mean tumor volumes were approx. 900-1200 mm³ (21 to 23 days post tumor cells injection).

Compound Formulation and Animal Treatment:

Compound A was prepared for dosing as homogenous suspensions in 1% carboxymethyl cellulose: 0.5% Tween® 80: 98.5% deionized water. Fresh suspensions were prepared once every 7 days and stored at 4° C. Compound A or vehicle was administered orally at a volume of 10 mL/kg.

Evaluation of Antitumor Activity:

Tumor volumes were measured with calipers and determined according to the formula: length×diameter²×π/6. In addition to presenting changes of tumor volumes over the course of treatments, antitumor activity is expressed as T/C % (mean change of tumor volume of treated animals/mean change of tumor volume of control animals)×100. Regressions (%) were calculated according to the formula ((mean tumor volume at end of treatment−mean tumor volume at start of treatment)/mean tumor volume at start of treatment)×100. Body weights and tumor volumes were recorded two to three times a week.

Blood Glucose Measurements Via Radio-Telemetry Technology (HD-XG Radio Telemetry Transmitter; Data Sciences International):

Blood glucose levels were measured continuously in conscious non-restrained freely moving rats by the method described in Brockway et al., Journal of Diabetes Science and Technology., 9(4):771-81 (2015), which is incorporated herein by reference in its entirety. Briefly, the 1.4 cc telemetry device provides direct continuous blood glucose readings along with temperature and activity for 4 weeks or longer. The device was used in non-tumor bearing Brown Norway (BN) rats. Each animal was surgically instrumented with glucose sensors in the abdominal aorta and the device placed in the intraperitoneal cavity. Continuous glucose readings were recorded with the Dataquest A.R.T. data acquisition system. Reference glucose values were measured from tail vein blood samples using the Nova StatStrip glucometer twice per week. Each animal was measured in cyclic runs of 1 minute for 10 seconds with a sampling rate of 1 Hz. Mean values for blood glucose levels, body temperature and motor activity were then computed and stored. Fifteen minutes or hourly averages were determined using the interval averaging routine on the Dataquest Analysis Software (Dataquest A.R.T, version 4.36; Data Sciences). Blood glucose values are expressed in mmol/L, body temperature in degree Celsius (° C.) and motor activity in number of movements (units) per minute.

Determination of Pharmacokinetic (PK) Parameters after Oral Administration of Compound A in Freely Moving Catheterized Rats Using Automated Blood Sampling (ABS) Technology:

The highly automated ABS system (Instech ABS2™) allows for unattended blood sample collection via an in-dwelling venous catheter placed in the jugular or femoral vein. For all animals, cannulas were filled with 1:1 heparin glycerol solution when not on study. The ABS freely-moving system is a well-recognized method to reduce stress during blood sampling and it only marginally impedes the animal in its freedom to move, drink, eat and sleep. Furthermore, this method allows obtaining pharmacokinetic parameters at night time (active phase of the animal).

Statistical Analysis:

Absolute values for primary tumor growth and body weight were used to make the statistical comparisons between groups (one way ANOVA followed by Dunnett's test for normally distributed data; ANOVA on Ranks for not normally distributed data followed by Dunnett's test for equal group size or Dunn's for unequal group size). Absolute values for blood glucose (calculated mean over 6 hours' time periods) and PK data were used to make the statistical comparisons between groups (two-tailed Student's t-tests). The significant level was set at p<0.05. All statistical calculations were carried out using SigmaStat.

### Results

Circadian Rhythms of Glucose and Motor Activity Measured in Conscious Unrestrained BN Rats:

A consistent diurnal rhythm of blood glucose level was observed (FIG. 1). Values were significantly lower (P<0.005) during the day (inactive phase) than during the night (active phase). A remarkable consistency in the pattern of diurnal variation of blood glucose levels (n=9) was observed for each of the 5 days of the experiment (FIG. 2).

Effects of Vehicle and Compound a Treatment on Blood Glucose Levels Measured in Conscious Unrestrained BN Rats:

Vehicle treatment at 10 AM (inactive phase) or 5 PM (active phase) had no effect on blood glucose levels (FIG. 3). At day 1 of treatment with Compound A at 10 AM (inactive phase) or 5 PM (active phase), a slight hyperglycemia was evidenced (FIG. 3). At steady state (Day 4-5 of daily treatment), a transient hyperglycemia profile was observed. Dosing before the inactive phase (10 a.m.) allowed blood glucose to normalize in between 2 doses, which could not be achieved when dosing before the active phase (5 p.m.). These observations could be confirmed when adding additional animals to our initial cohorts of rats (FIG. 7). After treatment discontinuation (recovery day 1) a significant transient hyperglycemia profile remained for a period up to 12h in the group dosed before the active phase (5 p.m.). In contrast blood glucose was already normalized to baseline levels at the start of recovery day 1 in the group dosed before the inactive phase (10 a.m., FIG. 7). Plasma PK profile assessed in conscious freely moving BN rats connected to an ABS system at day 1 or 4 (steady state) of treatment with Compound A at 10 AM (inactive phase) or 5 PM (active phase) did not revealed any significant differences (at 2, 4, 6, 8, 10, 12, 18 and 24h post treatment, FIG. 8).

Pk-Pd Modeling:

Phoenix WinNonlin 6.3 (Pharsight) was used to simulate the mean plasma concentration time profiles after multiple dosing using the non-compartmental nonparametric superposition approach of data generated from previous nude rats efficacy study. The predictions are based upon an accumulation ratio computed from the terminal slope (Lambda Z), allowing predictions from simple or complicated dosing schedules.

PK/PD Relationship at Steady State (Day 4) Following Compound A Treatment:

Compound A (50 mg/kg p.o. qd, n=6) treatment in BN rats induced a transient glucose level increase suggestive of glucose metabolism impairment consistent with hyperglycemia seen in patients treated with Compound A. This profile is reproducible over time (FIG. 3) and a PK/PD relationship based on modeled PK data in nude rats and measured glucose data in BN rats could be demonstrated (FIG. 4).

Case Study: 14 and 25 mg/kg Qd in “Alternative Schedule 1” Dosing Regimen in Nude Rats

Based upon the foregoing analysis, the pre-clinical blood glucose diurnal rhythms obtained for Compound A dosed either at 10 A.M. (during the inactive phase) or at 5 P.M. (during the active phase) described above would predict better tolerability of the following dosing schedule of Compound A: oral administration of Compound A once-per-day (q.d.) at 10 A.M. (inactive phase) for at least five-consecutive days. This alternative dosing schedule is referred to as “ALTERNATIVE SCHEDULE 1”. However, we wanted to confirm that the 10 A.M. (inactive phase) and 5 P.M. (active phase) dosing scheduling will not impair anti-tumor efficacy of Compound A. Thus we initiated 2 in-vivo efficacy experiments to address this question. As described herein, this model is here used to explore and guide dose scheduling in clinical studies.

FIG. 5 provides graphs showing the efficacy (left panel) of Compound A in Rat1-myr P110α tumor bearing nude rats treated orally with COMPOUND A at 14 mg/kg in ALTERNATIVE SCHEDULE 1 for 14 consecutive days as compared to 14 mg/kg qd dosed at 5 p.m. (i.e., during the active phase of the rat). No significant differences in tumor volume inhibition could be evidenced between the two scheduling's over the 2 weeks of continuous treatment. A very similar pattern was observed with body weight changes (right panel).

FIG. 6 provides the efficacy (left panel) of Compound A in Rat1-myr P110α tumor bearing nude rats treated orally with COMPOUND A at 25 mg/kg in ALTERNATIVE SCHEDULE 1 for 14 consecutive days as compared to 25 mg/kg qd dosed at 5 p.m. (i.e., during the active phase of the rat). No significant differences in tumor volume inhibition could be evidenced between the two scheduling's over the 2 weeks of continuous treatment. A very similar pattern was observed with body weight changes (right panel).

Based on our data, ALTERNATIVE SCHEDULE 1 for Compound A can achieve similar anti-tumor efficacy observed in nude rats orally administered Compound A once each day (q.d.) at 5 P.M. (active phase) on a continuous daily schedule at (a) 14 mg/kg, a dose which induces stasis and (b) at 25 mg/kg, a dose which achieve clear regression (50% tumor regression) following 2 weeks of treatment.

Assuming that the relationship between PD (glucose blood levels) and efficacy is similar in humans and tumor bearing rats, this model and analysis may be useful to predict host and tumor response in humans to ALTERNATIVE SCHEDULE 1.

IMPORTANT to notice: Given that the rats are nocturnal animals, their inactive phase applied with a ˜12-hour time difference to clinically active human subjects.
Case Study: 35 mg/kg Qd in “Alternative Schedule 1” Dosing Regimen in Combination with an Antiestrogen (Fulvestrant at 5 mg/kg s.c. Qw or Letrozole at 2.5 mg/kg p.o. Qd) in HBCx-19 and HBRX3077 (Both ER+/HER2-/PIK3CA Mutant PDX Breast Cancer) Sc Tumor Bearing Nude Mice

Based upon the foregoing analysis ALTERNATIVE SCHEDULE 1 for Compound A can achieve similar anti-tumor efficacy observed in nude rats orally administered Compound A either at 10 a.m. (inactive phase) or 5 P.M. (active phase). To confirm that the 10 A.M. (inactive phase) and 5 P.M. (active phase) dosing scheduling will not impair anti-tumor efficacy of Compound A. in combination with 2 different standard of cares (antiestrogen) in patient derived breast xenografts (PDX) tumor bearing nude mice, we initiated 3 in-vivo efficacy experiments. As described herein, this model is here used to explore and guide dose scheduling in clinical studies.

The experiment was conducted as described above and as further described in this Example.

Establishment of Patient-Derived Breast Xenograft (PDX) Models In Vivo:

PDX models were established by implanting surgical tumor tissues from treatment-naïve cancer patients into nude mice. All samples were anonymized and obtained with informed consent and under the approval of the institutional review boards of the tissue providers and Novartis. All PDX models were histologically characterized and independently confirmed for the external diagnosis and were genetically profiled using various technology platforms after serial passages in mice. PIK3CA mutation was determined by both RNA and DNA deep sequencing technologies and PIK3CA amplification was determined by SNP array 6.0. For efficacy studies, tumor-bearing animals were enrolled when subcutaneously implanted tumors reached about 200-300 mm³. HBCx-19 is an ER+Her2-negative luminal A tumor model with mutated PIK3CA. HBRX3077 is an ER+Her2-negative invasive ductal carcinoma tumor model with mutated PIK3CA.

Compound Formulation and Animal Treatment:

Compound A was prepared for dosing as homogenous suspensions in 1% carboxymethyl cellulose: 0.5% Tween® 80: 98.5% deionized water. Fresh suspensions were prepared once every 7 days and stored at 4° C. Compound A or vehicle was administered orally at a volume of 10 mL/kg.

Fulvestrant (Faslodex®, Astra Zeneca) stock solution at 50 mg/mL, was ready to use and stored at 4° C. in a light protected cabinet. It was administered subcutaneously once a week at a volume of 4 mL/kg.

Letrozole (Femara®, Novartis) 2.5 mg tablets were ready to use and stored at 4° C. in a light protected cabinet. It was administered orally daily as a suspension at a volume of 10 mL/kg.

FIGS. 9 and 10 respectively provide graphs showing the efficacy of Compound A in combination with Fulvestrant in HBCx-19 and HBRX3077 tumor bearing nude mice treated orally with COMPOUND A at 35 mg/kg (equivalent of the MTD of 400 mg QD in patients) in ALTERNATIVE SCHEDULE 1 for 21 (FIG. 9) or 17 (FIG. 10) consecutive days as compared to 35 mg/kg qd dosed at 5 p.m. (i.e., during the active phase of the mice). No significant differences in tumor volume inhibition could be evidenced between the two scheduling's over the 2-3 weeks of continuous treatment. A very similar pattern was observed with body weight changes (data not shown).

FIG. 11 provides graphs showing the efficacy of Compound A in combination with Letrozole in HBRX3077 tumor bearing nude mice treated orally with COMPOUND A at 35 mg/kg in ALTERNATIVE SCHEDULE 1 for 17 consecutive days as compared to 35 mg/kg qd dosed at 5 p.m. (i.e., during the active phase of the mice). No significant differences in tumor volume inhibition could be evidenced between the two scheduling's over the 2-3 weeks of continuous treatment. A very similar pattern was observed with body weight changes (data not shown).

Based on the foregoing data, ALTERNATIVE SCHEDULE 1 for Compound A combined with the antiestrogen agents fulvestrant or letrozole can achieve similar anti-tumor efficacy observed in nude mice orally administered Compound A once each day (q.d.) at 5 P.M. (active phase) on a continuous daily schedule at 35 mg/kg, a dose which achieve clear regression (35 to 50% tumor regression in 2 out of 3 model tested) following 17 days of treatment.

Assuming that the relationship between PD (glucose blood levels) and efficacy is similar in humans and tumor bearing mice, this model and analysis may be useful to predict host and tumor response in humans to ALTERNATIVE SCHEDULE 1. IMPORTANT to notice: Given that the mice are nocturnal animals, their inactive phase applied with a ˜12-hour time difference to clinically active human subjects.

1. A method of treating or preventing a proliferative disease in a patient in need thereof, comprising administering a therapeutically effective amount of a phosphatidylinositol 3-kinase inhibitor selected from the compound of formula (I) 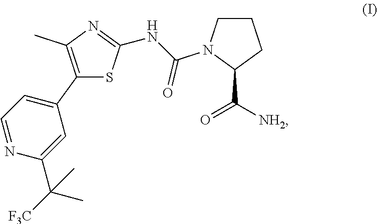 [SMILES: Cc1nc(NC(=O)N2CCC[C@H]2C(N)=O)sc1-c1ccnc(C(C)(C)C(F)(F)F)c1]
the compound of formula (II) 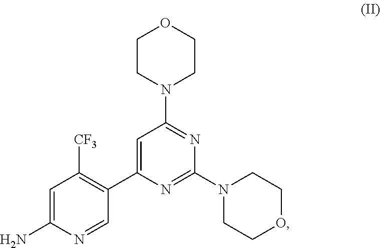 [SMILES: Nc1cc(C(F)(F)F)c(-c2cc(N3CCOCC3)nc(N3CCOCC3)n2)cn1]
pictilisib, taselisib, LY2780301, copanlisib, MLN1117, and AZD8835 and a pharmaceutically acceptable salt thereof to the patient once-per-day either on a continuous daily schedule or an intermittent schedule at about zero to about three hours prior to sleep.

2. (canceled)

3. The method of claim 1, wherein the phosphatidylinositol 3-kinase inhibitor is the compound of formula (I) 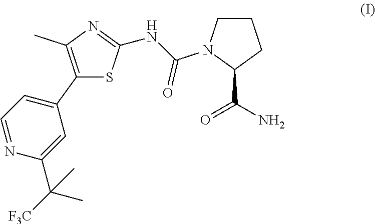 [SMILES: Cc1nc(NC(=O)N2CCC[C@H]2C(N)=O)sc1-c1ccnc(C(C)(C)C(F)(F)F)c1]
or a pharmaceutically acceptable salt thereof and administered orally in a therapeutically effective amount of about 50 mg to about 450 mg once-per-day either on a continuous daily schedule or an intermittent schedule.

4. (canceled)

5. The method of claim 1, wherein the phosphatidylinositol 3-kinase inhibitor is administered at about one to about two hours prior to sleep.

6. The method of claim 1, wherein the phosphatidylinositol 3-kinase inhibitor is administered at night.

7. The method of claim 1, wherein the phosphatidylinositol 3-kinase inhibitor is administered with food at about one to three hours prior to sleep.

8. The method of claim 7, wherein the phosphatidylinositol 3-kinase inhibitor is administered within about zero to about one hour of ingesting food.

9. The method of claim 1, further comprising administering the phosphatidylinositol 3-kinase inhibitor on a continuous daily schedule.

10. (canceled)

11. A method of treating or preventing a proliferative disease comprising first administering to a patient in need thereof a therapeutically effective amount of a phosphatidylinositol 3-kinase inhibitor selected from the compound of formula (I) 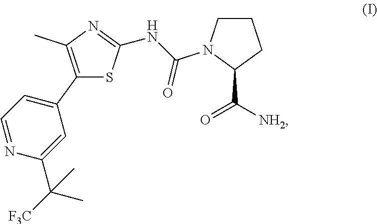 [SMILES: Cc1nc(NC(=O)N2CCC[C@H]2C(N)=O)sc1-c1ccnc(C(C)(C)C(F)(F)F)c1]
the compound of formula (II) 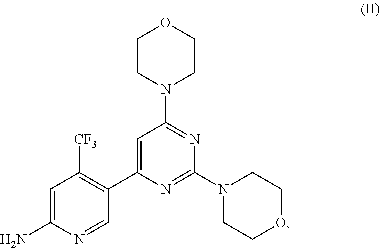 [SMILES: Nc1cc(C(F)(F)F)c(-c2cc(N3CCOCC3)nc(N3CCOCC3)n2)cn1]
pictilisib, taselisib, LY2780301, copanlisib, MLN1117, and AZD8835 and a pharmaceutically acceptable salt thereof once in each morning or twice daily; second determining said patient has a side effect of hyperglycemia after administration of said phosphatidylinositol 3-kinase inhibitor to said patient; and third shifting the administration of the phosphatidylinositol 3-kinase inhibitor to once-per-day either on a continuous daily schedule or an intermittent schedule at about zero to about three hours prior to sleep.

12. (canceled)

13. The method of claim 12, wherein the phosphatidylinositol 3-kinase inhibitor is the compound of formula (I) 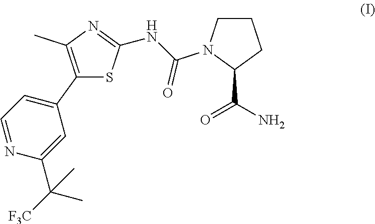 [SMILES: Cc1nc(NC(=O)N2CCC[C@H]2C(N)=O)sc1-c1ccnc(C(C)(C)C(F)(F)F)c1]
or a pharmaceutically acceptable salt thereof and administered orally in a therapeutically effective amount of about 50 mg to about 450 mg per day.

14-18. (canceled)

19. A method according to claim 1, wherein the proliferative disease is a cancer.

20. A method according to claim 1, wherein the proliferative disease is a cancer selected from a cancer of the lung, bronchus, prostate, breast (including sporadic breast cancers and sufferers of Cowden disease), colon, rectum, colon carcinoma, colorectal adenoma, pancreas, gastrointestine, hepatocellular, stomach, gastric, ovary, squamous cell carcinoma, and head and neck.

21. A method according to claim 1, wherein the proliferative disease is breast cancer.

22. A method according to claim 1, wherein the phosphatidylinositol 3-kinase inhibitor, or a pharmaceutically acceptable salt thereof, is administered in combination with at least one additional therapeutic agent.

23. A therapeutic regimen for the treatment or prevention of a proliferative disease comprising administering a therapeutically effective amount of a phosphatidylinositol 3-kinase inhibitor selected from the compound of formula (I) 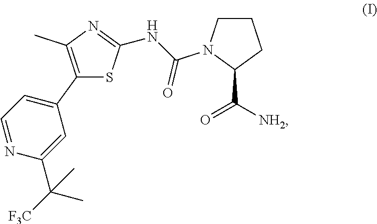 [SMILES: Cc1nc(NC(=O)N2CCC[C@H]2C(N)=O)sc1-c1ccnc(C(C)(C)C(F)(F)F)c1]
the compound of formula (II) 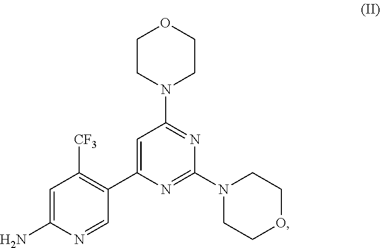 [SMILES: Nc1cc(C(F)(F)F)c(-c2cc(N3CCOCC3)nc(N3CCOCC3)n2)cn1]
pictilisib, taselisib, LY2780301, copanlisib, MLN1117, and AZD8835 and a pharmaceutically acceptable salt thereof once-per-day either on a continuous daily schedule or an intermittent schedule at about zero to about three hours prior to sleep.

24-27. (canceled)
